(19) United States
(12) Patent Application Publication
(10) Pub. No.: US 2011/0207671 A1
(43) Pub. Date: Aug. 25, 2011

(54) METHOD FOR PRODUCING DOUBLE-CROSSLINKED COLLAGEN

(75) Inventors: Robert C. Chang, Burlingame, CA (US); David R. Olsen, Menlo Park, CA (US); Jame W. Polarek, Sausalito, CA (US); Kim E. Williams, San Carlos, CA (US)

(73) Assignee: FibroGen, Inc., San Francisco, CA (US)

(21) Appl. No.: 12/737,828

(22) PCT Filed: Aug. 21, 2009
(86) PCT No.: PCT/US2009/004774

§ 371 Date: Apr. 25, 2011

Related U.S. Application Data
(60) Provisional application No. 61/189,988, filed on Aug. 22, 2008.

(51) Int. Cl. A61K 8/65; C07K 1/107; C07K 14/78; A61Q 19/00

(52) U.S. Cl. 514/17.2; 530/356

(57) ABSTRACT

The present invention relates to double-crosslinked collagen materials, methods for preparing double-crosslinked collagen materials, and methods of using double-crosslinked

This application claims the benefit of U.S. Provisional Application Ser. No. 61/189,988, filed on 22 Aug. 2008, which is incorporated by reference herein it its entirety.

### FIELD OF THE INVENTION

The present invention relates to double-crosslinked collagen materials, methods for preparing double-crosslinked collagen materials, and methods of using double-crosslinked collagen materials.

### BACKGROUND OF THE INVENTION

Crosslinking of collagen is an effective method to modify the stability of collagen compositions and materials and to optimize their mechanical and structural properties. Crosslinked collagen materials are used extensively in various medical and industrial applications. For example, crosslinked collagen materials are used to replace or augment hard or soft connective tissue, such as skin, tendons, cartilage, bone, and interstitium. Crosslinked collagen materials have been implanted surgically, and numerous injectable crosslinked collagen formulations are currently available for various cosmetic applications.

Generally, glutaraldehyde has been used as a crosslinking agent to crosslink naturally-derived collagens (i.e., collagen obtained by extraction from the connective tissues of animals (e.g., bovine and porcine skin, bone, and cartilage)). Alternative crosslinking methods for collagen have been described, including the use of bifunctional or multifunctional crosslinking agents, such as diisocyanates and epoxy compounds, which bridge amine groups between adjacent polypeptide chains. Additionally, other crosslinking agents which activate carboxylic acid groups of glutamic acid or aspartic acid residues to react with amine groups on another polypeptide chain have been used to crosslink naturally-derived collagens.

Current methods for preparing crosslinked collagen materials usually employ a single crosslinking agent, resulting in a single-crosslinked collagen material. The single-crosslinked collagen materials obtained have physical properties that can limit their use in particular applications. For example, the persistence of commercially available naturally-derived single-crosslinked collagen materials is limited by the degradation and resorption of the material following implantation, often requiring patients to undergo retreatment. There thus remains a need in the art for methods of producing crosslinked collagen materials having improved physical properties (e.g., enhanced persistence).

Current methods for preparing crosslinked collagen materials can also result in a poor yield of crosslinked material. There thus remains a need in the art for methods of producing crosslinked collagen materials having an improved yield

The present invention meets one or more of these needs by providing methods for preparing double-crosslinked collagen materials, double-crosslinked collagen materials, and methods of using double-crosslinked collagen materials.

### SUMMARY OF THE INVENTION

The present invention provides methods for producing double-crosslinked collagen material comprising: providing a collagen starting material, a first crosslinking agent, and a second crosslinking agent; subjecting the collagen and the first crosslinking agent to a first crosslinking reaction, wherein the first crosslinking reaction is performed under reaction conditions that allow the first crosslinking reaction to occur, thereby obtaining a single-crosslinked collagen material; and subjecting the single-crosslinked collagen material to a second crosslinking reaction using the second crosslinking agent, wherein the second crosslinking agent is not the same as the first crosslinking agent, and wherein the second crosslinking reaction is performed under reaction conditions that allow the second crosslinking reaction to occur, thereby obtaining a double-crosslinked collagen material.

The reaction conditions (e.g., pH, temperature, crosslinking reaction time, concentration of crosslinking agents, etc.) used for producing double-crosslinked collagen materials of the present invention, including the reaction conditions used for the first crosslinking reaction and the reaction conditions used for the second crosslinking reaction, may vary depending upon the specific type of crosslinking agent used, the extent of crosslinking desired, or the type of collagen used as the collagen starting material. Accordingly, in various embodiments, the reaction conditions comprise a temperature between about 20-50° C. In other embodiments, the reaction conditions comprise a pH between about 7-10. In yet other embodiments, the reaction conditions comprise a time between about 1 to 16 hours. In other embodiments, the concentration of the crosslinking agent is between about 0.0003-4%.

The present invention provides methods for increasing the recovery yield of crosslinked collagen materials. In certain embodiments, the yield of double-crosslinked collagen material produced using the methods of the present invention is about 65-100%, about 70-100%, about 75-100%, about 80-100%, about 85-100%, about 90-100%, about 95-100%, about 75-99%, about 75-95%, about 75-90%, about 75-85%, or about 75-80%. In particular embodiments, the yield of double-crosslinked collagen materials produced using methods of the present invention is about 75-90%.

In some embodiments, the first crosslinking agent used in the first crosslinking reaction is an aldehyde compound, a carbodiimide compound, or an epoxide compound and the second crosslinking agent used in the second crosslinking reaction is an aldehyde, a carbodiimide, or an epoxide compound.

In some embodiments, the first crosslinking agent used in the first crosslinking reaction is not the same as the second crosslinking agent used in the second crosslinking reaction. For example, in one embodiment, the first crosslinking agent used in the first crosslinking reaction is an aldehyde compound and the second crosslinking agent used in the second crosslinking reaction is a carbodiimide or an epoxide compound. In another embodiment, the first crosslinking agent used in the first crosslinking reaction is a carbodiimide compound and the second crosslinking agent used in the second crosslinking reaction is an epoxide or an aldehyde compound. In yet another embodiment, the first crosslinking agent used in the first crosslinking reaction is an epoxide compound and the second crosslinking agent used in the second crosslinking reaction is a carbodiimide or an aldehyde.

In certain embodiments, the first crosslinking agent is a zero-length crosslinker or a homobifunctional crosslinker. In other embodiments, the first crosslinking agent is an aldehyde, a carbodiimide, or an epoxide compound. Similarly, the second crosslinking agent may be an aldehyde, a carbodiimide, or an epoxide compound. In one embodiment, the aldehyde is formaldehyde, glyoxal, malondialdehyde, succinaldehyde, glutaraldehyde, or adipaldehyde. In another embodiment, the carbodiimide is 1-ethyl-3-(3-dimethylaminopropyl)carbodiimide hydrochloride (EDC), 1-Cyclohexyl-3-(2-morpholinoethyl)carbodiimide (CMC), dicyclohexyl carbodiimide (DCC), or diisopropyl carbodiimide (DIC). In yet another embodiment, the epoxide is 1,4-butanediol diglycidyl ether (BDDE), ethylene glycol diglycidyl ether (EGDGE), 1,6-hexanediol diglycigyl ether, polyethylene glycol diglycidyl ether, polypropylene glycol diglycidyl ether, polytetramethylene glycol digylcidyl ether, neopentyl glycol digylcidyl ether, polyglycerol polyglycidyl ether, diglycerol polyglycidyl ether, glycerol polyglycidyl ether, trimethylolpropane polyglycidyl ether, pentaerythritol polyglycidyl ether, sorbitol polyglycidyl ether, sorbitan polygycidyl ether, resorcin diglycidyl ether, glycerol polyglycidyl ether (EX-313 EC), (EX-314 EC), or (EX-810 EC).

In some embodiments, the crosslink initiated by the first crosslinking agent occurs by the reaction of the crosslinking agent with collagen α-amine groups of either lysine or hydroxylysine residues. In these embodiments, the first crosslinking agent may in particular be an aldehyde compound, e.g. glutaradehyde. In these and other embodiments, the crosslink initiated by the second crosslinking agent may also occur by the reaction of the crosslinking agent with collagen α-amine groups of either lysine or hydroxylysine residues. In these embodiments, the second crosslinking agent may in particular be an epoxide compound, e.g. BDDE.

The collagen starting material used for producing double-crosslinked collagen material of the present invention is a collagen or collagens of any type. In certain embodiments, the double-crosslinked collagen material of the present invention is produced from a collagen starting material comprising a fibril forming collagen (e.g., type I, type II, type III, type V, or type XI collagen). An example of a suitable collagen starting material is SEQ ID NO:1, which is the amino acid sequence of al type III collagen. In other embodiments, the double-crosslinked collagen material of the present invention is produced from a collagen starting material comprising a fibril associated collagen (e.g., type IX, type XII, type XIV, type XVI, type XIX, or type XXI collagen). In yet other embodiments, the double-crosslinked collagen material of the present invention is produced from a collagen starting material comprising a sheet forming collagen (e.g., type IV, type VIII, or type X collagen). In further embodiments, the double-crosslinked collagen material of the present invention is produced from a collagen starting material comprising a beaded filament collagen or an anchoring fibril collagen (e.g., type VI collagen and type VII collagen, respectively). Other collagen types useful in the present methods include type XIII, type XV, type XVII, type XVIII, type XX, type XXII, type XXIII, type XXIV, type XXV, type XXVI, type XXVII, and type XXVIII collagen. In a particular embodiment, a fibril forming collagen (i.e., type I, type II, type III, type V, or type XI collagen) is the collagen starting material used to produce double-crosslinked collagen according to the methods of the present invention.

In one embodiment, the collagen starting material useful for producing double-crosslinked collagen material is recombinant collagen. In another embodiment, the collagen starting material useful for producing double-crosslinked collagen material is recombinant human collagen. The use of any single type of recombinant collagen (e.g., recombinant type I collagen, recombinant type II collagen, recombinant type III collagen, etc.) or any mixture of more than one type of recombinant collagen (e.g., a mixture of recombinant type I collagen and recombinant type III collagen) as the collagen starting material for producing a double-crosslinked collagen material is specifically provided by the present invention.

When a fibril forming collagen is used as the collagen starting material, the collagen may be formed into fibrils prior to subjecting the collagen and the first crosslinking agent to the first crosslinking reaction. If the collagen has been obtained from a natural source, then this fibril formation may have taken place in vivo. However, fibril formation may also be carried out in vitro, particularly when the collagen starting material is recombinant collagen. Methods of forming fibrils are known in the art. (See, e.g., Williams et al. (1978) J Biol Chem. 253:6578-6585; McPherson et al. (1985) Coll Relat Res. 5:119-35; Birk et al. (1984) Arch Biochem Biophys. 235:178-85.) Recombinant collagen may be formed into fibrils by placing the collagen in a fibrillogenesis buffer. An example of a suitable fibrillogenesis buffer is 0.2 M NaPO₄, pH 11.2.

A collagen starting material useful for producing a double-crosslinked collagen material according to the present invention is type III collagen. In one embodiment, the collagen starting material for use in the present methods is type III collagen having an amino acid sequence of SEQ ID NO:1 or a collagenous fragment thereof. In another embodiment, the collagen starting material for use in the present methods comprises a collagen having an amino acid sequence of amino acid residue 168 to amino acid residue 1196 of SEQ ID NO:1. In yet another embodiment, the collagen starting material for use in the present methods has an amino acid sequence of from amino acid residue 168 to amino acid residue 1196 of SEQ ID NO:1.

In another embodiment, the collagen starting material for use in the present methods is a collagen having an amino acid sequence of SEQ ID NO:2 or a collagenous fragment thereof. In one embodiment, the collagen starting material for use in the present methods comprises a collagen having an amino acid sequence of from amino acid residue 38 to amino acid residue 1066 of SEQ ID NO:2. In another embodiment, the collagen starting material for use in the present methods has an amino acid sequence of from amino acid residue 38 to amino acid residue 1066 of SEQ ID NO:2.

Other collagens can be used as the collagen starting material for producing double-crosslinked collagen materials according to the methods of the present invention. In one embodiment, the collagen starting material for use in the present methods is a collagen having an amino acid sequence of SEQ ID NO:2, wherein the amino acid sequence contains an isoleucine to proline substitution at amino acid residue 822 of SEQ ID NO:2. In another embodiment, the collagen starting material for use in the present methods is a collagen having an amino acid sequence of from amino acid residue 38 to amino acid residue 1066 of SEQ ID NO:2, wherein the amino acid sequence contains an isoleucine to proline substitution at amino acid residue 822 of SEQ ID NO:2 (collagen type III-A).

In another embodiment, the collagen starting material for use in the present methods is a collagen having an amino acid sequence of SEQ ID NO:2, wherein the amino acid sequence contains proline substitutions at amino acid residues 817, 820, 823, 826, and 829 of SEQ ID NO:2. In another embodiment, the collagen starting material for use in the present methods is a collagen having an amino acid sequence of from amino acid residue 38 to amino acid residue 1066 of SEQ ID NO:2, wherein the amino acid sequence contains proline substitutions at amino acid residues 817, 820, 823, 826, and 829 of SEQ ID NO:2 (collagen type III-B).

In yet another embodiment, the collagen starting material for use in the present methods is a collagen having an amino acid sequence of SEQ ID NO:2, wherein the amino acid sequence contains proline substitutions at amino acid residues 817, 820, 822, 823, 826, and 829 of SEQ ID NO:2. In another embodiment, the collagen starting material for use in the present methods is a collagen having an amino acid sequence of from amino acid residue 38 to amino acid residue 1066 of SEQ ID NO:2, wherein the amino acid sequence contains proline substitutions at amino acid residues 817, 820, 822, 823, 826, and 829 of SEQ ID NO:2 (collagen type III-C).

In yet another embodiment, the collagen starting material for use in the present methods is a collagen having an amino acid sequence of SEQ ID NO:2, wherein the amino acid sequence contains proline substitutions at amino acid residues 265, 300, 402, 414, 468, 471, 543, 567, 576, 603, 618, 693, 717, 738, and 900 of SEQ ID NO:2. In another embodiment, the collagen starting material for use in the present methods is a collagen having an amino acid sequence of from amino acid residue 38 to amino acid residue 1066 of SEQ ID NO:2, wherein the amino acid sequence contains proline substitutions at amino acid residues 265, 300, 402, 414, 468, 471, 543, 567, 576, 603, 618, 693, 717, 738, and 900 of SEQ ID NO:2 (collagen type III-D).

In certain embodiments of the present invention, the collagen starting material used for producing double-crosslinked collagen materials is a collagen free of intramolecular crosslinks, free of intermolecular crosslinks, free of endogenous crosslinks, free of propeptide sequence, free of telopeptide sequence (i.e., atelopeptide collagen), or free of hydroxylation, including, for example, free of proline hydroxylation. In particular embodiments, the collagen starting material used for producing double-crosslinked collagen materials is a recombinant collagen, including a recombinant human collagen, wherein the recombinant collagen or recombinant human collagen is free of intramolecular crosslinks, free of intermolecular crosslinks, free of endogenous crosslinks, free of propeptide sequence, free of telopeptide sequence, or free of hydroxylation, including free of proline hydroxylation.

The present invention provides double-crosslinked collagen materials. In some embodiments, the double-crosslinked collagen material of the present invention has an extent of crosslinking between about 65% and 100%. In other embodiments, the double-crosslinked collagen material has a melting temperature between about 65° C. and 80° C. In further embodiments, the double-crosslinked collagen material of the present invention has active pendant epoxy groups between about 0.5 and 2 moles.

In one embodiment, the present invention provides an implantable collagen composition comprising a double-crosslinked collagen material. In another embodiment, the present invention provides an implantable collagen composition comprising a double-crosslinked collagen material produced by the methods of the present invention. In one aspect, the double-crosslinked collagen materials of the present invention have enhanced in vivo persistence relative to single-crosslinked or non-crosslinked collagen materials. In another aspect, the double-crosslinked collagen materials of the present invention have decreased immunogenicity relative to single-crosslinked or non-crosslinked collagen materials.

The present invention provides a kit useful for augmenting, bulking, or replacing tissue of a mammal, the kit comprising a double-crosslinked collagen material produced by the methods of the present invention and a label with instructions for administering the double-crosslinked collagen material. In another embodiment, the present invention provides a kit useful for augmenting soft tissue, the kit comprising a double-crosslinked collagen material produced by the methods of the present invention, a syringe, and a needle.

The double-crosslinked collagen materials produced by the methods of the present invention may be used in the preparation of a product for pharmaceutical, cosmetic, or medical use. Double-crosslinked collagen materials produced by methods of the invention are suitable for use in therapy or surgery. Double-crosslinked collagen materials produced by the methods of the invention are suitable for use in tissue augmentation or repair. In one embodiment, the present invention provides a cosmetic procedure comprising injecting or implanting a double-crosslinked collagen material produced by the method of the invention into the skin or dermis of a subject. In some embodiments, the present invention provides a method for augmenting, bulking, or replacing tissue of a mammal comprising administering the double-crosslinked collagen materials produced by the methods of the present invention to tissue of a mammal. In one aspect the double-crosslinked collagen material is administered by injection.

In a further embodiment, the present invention provides novel compositions comprising collagen, wherein the collagen is a recombinant type III collagen. In one aspect, the recombinant type III collagen comprises amino acid residue 38 to amino acid residue 1066 of SEQ ID NO:2, wherein the amino acid sequence contains proline substitutions at amino acid residues 817, 820, 823, 826, and 829 of SEQ ID NO:2. In another aspect, the recombinant type III collagen comprises amino acid residue 38 to amino acid residue 1066 of SEQ ID NO:2, wherein the amino acid sequence contains proline substitutions at amino acid residues 817, 820, 822, 823, 826, and 829 of SEQ ID NO:2. In yet another aspect, the recombinant type III collagen comprises amino acid residue 38 to amino acid residue 1066 of SEQ ID NO:2, wherein the amino acid sequence contains proline substitutions at amino acid residues 265, 300, 402, 414, 468, 471, 543, 567, 576, 603, 618, 693, 717, 738, and 900 of SEQ ID NO:2.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items. It must be noted that as used herein and in the appended claims, the singular forms “a”, “an”, and “the” include plural references unless context clearly dictates otherwise. Thus, for example, a reference to a “collagen” or “collagen material” or “collagen starting material” may be a reference to one or more collagen types.

### DESCRIPTION OF THE INVENTION

The present invention relates in some aspects to the discovery that collagen crosslinked in a sequential manner by two different crosslinking agents results in a double-crosslinked collagen material having improved performance and production/recovery characteristics. Double-crosslinked collagen materials, and methods for producing double-crosslinked collagen materials, are provided. Various uses of the double-crosslinked collagen materials in pharmaceutical, medical, and cosmetic applications, including, for example, tissue augmentation, are also provided herein.

Although not intending to be bound by any particular theory of operation, it is believed that the first crosslinking reaction introduces a sufficient number of crosslinks into the collagen material to prevent or substantially reduce the dissolution of collagen fibrils at the higher pH conditions used in the second crosslinking reaction. Since a decrease in the dissolution of collagen fibrils during crosslinking reactions will increase the amount of collagen material recovered, the methods of the present invention may increase the yield of crosslinked collagen material.

The section headings are used herein for organizational purposes only, and are not to be construed as in any way limiting the subject matter described herein.

Methods for Producing Double-Crosslinked Collagen Materials

The present invention provides methods for producing double-crosslinked collagen materials.

Generally, methods for producing double-crosslinked collagen material according to the present invention comprise: providing a collagen starting material (e.g., collagen fibrils), a first crosslinking agent, and a second crosslinking agent; subjecting the collagen starting material and the first crosslinking agent to a first crosslinking reaction, wherein the first crosslinking reaction is performed under reaction conditions (e.g., within a particular pH range) that allow the first crosslinking reaction to occur, thereby obtaining a single-crosslinked collagen material; and subjecting the single-crosslinked collagen material to a second crosslinking reaction using the second crosslinking agent, wherein the second crosslinking agent is not the same as the first crosslinking agent, and wherein the second crosslinking reaction is performed under reaction conditions (e.g., within a particular pH range) that will allow the second crosslinking reaction to occur, thereby obtaining a double-crosslinked collagen material.

The reaction conditions (e.g., pH, temperature, crosslinking reaction time, concentration of collagen, concentration of crosslinking agents, etc) used for producing double-crosslinked collagen materials of the present invention, including the reaction conditions used for the first crosslinking reaction and the reaction conditions used for the second crosslinking reaction, may vary depending upon the specific type of crosslinking agent used, the extent of crosslinking desired, or the type of collagen used as the collagen starting material. One of ordinary skill in the art can empirically determine appropriate reaction conditions for producing double-crosslinked collagen materials according to the present invention without necessitating undue experimentation. Combined with the teachings provided herein, by choosing among the various available collagen types, crosslinking agents, and reaction conditions (e.g., pH, temperature, time, concentration), a person skilled in the art is able to produce double-crosslinked collagen materials according to the present invention.

In some embodiments, the first crosslinking agent used in the first crosslinking reaction is not the same as the second crosslinking agent used in the second crosslinking reaction. As a non-limiting example, in carrying out the sequential double crosslinking methods according to the present invention, the first crosslinking agent used in the first crosslinking reaction can be an aldehyde compound, in which case the second crosslinking agent used in the second crosslinking reaction can be a carbodiimide or an epoxide compound. Alternatively, if the first crosslinking agent used in the first crosslinking reaction is a carbodiimide compound, the second crosslinking agent used in the second crosslinking reaction can be an epoxide or an aldehyde compound. Additionally, if the first crosslinking agent used in the first crosslinking reaction is an epoxide compound, the second crosslinking agent used in the second crosslinking reaction can be a carbodiimide or an aldehyde. Briefly, the order for using any crosslinking agent (e.g., carbodiimide, an aldehyde, or an epoxide compound) to perform the two crosslinking reactions is interchangeable.

Each crosslinking reaction (i.e., the first crosslinking reaction or the second crosslinking reaction) may be carried out at a temperature according to the judgment of those of skill in the art. In certain embodiments; each crosslinking reaction is carried out at about 0-50° C., about 20-50° C., about 20-45° C., about 20-40° C., about 20-35° C., or about 20-30° C. In other embodiments, each crosslinking reaction is carried out at about 0° C., about 5° C., about 10° C., about 15° C., about 20° C., about 25° C., about 30° C., about 35° C., about 40° C., about 45° C., or about 50° C. In particular embodiments, each crosslinking reaction is carried out at about 20-40° C.

Each crosslinking reaction (i.e., the first crosslinking reaction or the second crosslinking reaction) may be carried out at a pH according to the judgment of those of skill in the art. For example, it is well-known in the art that crosslinking agents are effective at crosslinking at a particular pH or ranges of pH. Therefore, depending on which type of crosslinking agent is used for any of the crosslinking reactions, one of ordinary skill in the art can choose an appropriate pH or range of pH that will be effective to allow a crosslinking reaction to occur. In certain embodiments, each crosslinking reaction is carried out at a pH of about 6-12, about 7-12, about 7-11, about 7-10, or about 7.2-10. In other embodiments, each crosslinking reaction is carried out at a pH of about 6, about 7, about 7.2, about 9, about 10, about 11, or about 12. In particular embodiments, each crosslinking reaction is carried out at a pH of about 7-10. In one embodiment, the second crosslinking reaction is carried out at a higher (i.e. more basic) pH than the first crosslinking reaction. Typically, the second crosslinking reaction is carried out at a basic pH, e.g. a pH of about 7-12, about 7-11, about 7-10, or about 7.2-10.

Each crosslinking reaction (i.e., the first crosslinking reaction or the second crosslinking reaction) may be carried out for a period of time according to the judgment of those of skill in the art. In certain embodiments, each crosslinking reaction is carried out for about 1 minute to 72 hours, about 1-72 hours, about 3-72 hours, about 4-72 hours, about 4-48 hours, about 4-40 hours, about 4-24 hours, or about 4-16 hours. In certain embodiments, each crosslinking reaction is carried out for about 1 minute, about 30 minutes, about 1 hour, about 2 hours, about 3 hours, about 4 hours, about 5 hours, about 10 hours, about 16 hours, about 20 hours, about 24 hours, about 40 hours, about 48 hours, or about 72 hours. In particular embodiments, each crosslinking reaction is carried out for 16 hours.

The concentration of crosslinking agent used in each crosslinking reaction (i.e., the first crosslinking reaction or the second crosslinking reaction) may be a concentration according to the judgment of those of skill in the art. In certain embodiments, the concentration of the crosslinking agent is about 0.0001-10%, about 0.0005-0.5%, about 0.001-0.5%, about 0.002-0.5%, about 0.004-0.5%, about 0.005-0.5%, about 0.01-0.5%, about 0.05-0.5%, about 0.1-0.5%, about 0.5-1%, about 0.75-1%, about 1-10%, about 1-5%, about 1-4%, about 1-2.5%, or about 1-2%. In particular embodiments, the concentration of the crosslinking agent is about 0.0035%. In other embodiments, the concentration of the crosslinking agent is about 4%.

The methods of the present invention result in double-crosslinked collagen materials with increased recovery yield. The recovery yield can be determined by various methods available to one of skill for determining recovery yield. For example, in one embodiment, recovery yield is determined as the ratio of the final amount of double-crosslinked collagen material produced to the amount of collagen starting material. In certain embodiments, the yield of double-crosslinked collagen material produced using methods of the present invention will be about 38-100%, about 40-100%, about 45-100%, about 50-100%, about 55-100%, about 60-100%, about 65-100%, about 70-100%, about 75-100%, about 80-100%, about 85-100%, about 90-100%, about 95-100%, about 75-99%, about 75-95%, about 75-90%, about 75-85%, or about 75-80%. In particular embodiments, the yield of double-crosslinked collagen material produced using methods of the present invention will be about 75-90%.

Collagens for Use in the Present Methods

The collagen starting material used for producing double-crosslinked collagen material of the present invention can be a collagen or collagens of any type. In certain embodiments, the double-crosslinked collagen material of the present invention is produced from a collagen starting material comprising a fibril forming collagen. Fibril forming collagens include type I, type II, type III, type V, and type XI collagens. In other embodiments, the double-crosslinked of the present invention is produced from a collagen starting material comprising a fibril associated collagen. Fibril associated collagens include type IX, type XII, type XIV, type XVI, type XIX, and type XXI collagens. In other embodiments, the double-crosslinked collagen material of the present invention is produced from a collagen starting material comprising a sheet forming collagen. Sheet forming collagens include type IV, type VIII, and type X collagens. In yet other embodiments, the double-crosslinked collagen material of the present invention is produced from a collagen starting material comprising a beaded filament collagen or an anchoring fibril collagen. Beaded filament collagens and anchoring filament collagens include type VI collagen and type VII collagen, respectively. Other collagen types useful in the present methods include type XIII, type XV, type XVII, type XVIII, type XX, type XXII, type XXIII, type XXIV, type XXV, type XXVI, type XXVII, and type XXVIII collagen. (See Haralson and Hassell, Extracellular Matrix, A Practical Approach, 8-11, Oxford University Press, 1995, the contents of which is hereby incorporated by reference in its entirety.) In a particular embodiment, a fibril forming collagen (i.e., type I, type II, type III, type V, or type XI collagen) is the collagen starting material used to produce double-crosslinked collagen according to the methods of the present invention.

In one embodiment, the collagen starting material useful for producing double-crosslinked collagen material is recombinant collagen. In another embodiment, the collagen starting material useful for producing double-crosslinked collagen material is recombinant human collagen. The use of any single type of recombinant collagen (e.g., recombinant type I collagen, recombinant type II collagen, recombinant type III collagen, etc.) or any mixture of more than one type of recombinant collagen (e.g., a mixture of recombinant type I collagen and recombinant type III collagen) as the collagen starting material for producing a double-crosslinked collagen material is specifically contemplated by the present invention. Recombinant collagens and methods of their production have been described in, e.g., International Publication Nos. WO 2006/052451 and WO 1993/007889, each of which is hereby incorporated by reference in its entirety.

A collagen starting material useful for producing a double-crosslinked collagen material according to the present invention is type III collagen. In one embodiment, the collagen starting material for use in the present methods is type III collagen having an amino acid sequence of SEQ ID NO:1 or a collagenous fragment thereof. The N-propeptide domain of type III collagen is from amino acid residue 24 to amino acid residue 153 of SEQ ID NO:1. The N-telopeptide domain of type III collagen is from amino acid residue 154 to amino acid residue 167 of SEQ ID NO:1. The α-helical domain of type III collagen is from amino acid residue 168 to amino acid residue 1196 of SEQ ID NO:1. The C-telopeptide of type III collagen is from amino acid residue 1197 to amino acid residue 1221 of SEQ ID NO:1. The C-propeptide of type III collagen is from amino acid residue 1222 to amino acid residue 1466 of SEQ ID NO:1. In one embodiment, the collagen starting material for use in the present methods comprises a collagen having an amino acid sequence of amino acid residue 168 to amino acid residue 1196 of SEQ ID NO:l. In another embodiment, the collagen starting material for use in the present methods has an amino acid sequence of from amino acid residue 168 to amino acid residue 1196 of SEQ ID NO:1.

In another embodiment, the collagen starting material for use in the present methods is a collagen having the amino acid sequence of SEQ ID NO:2 or a collagenous fragment thereof. In the collagen having an amino acid sequence of SEQ ID NO:2, the N-telopeptide domain is from amino acid residue 24 to amino acid residue 37 of SEQ ID NO:2; the α-helical domain is from amino acid residue 38 to amino acid residue 1066 of SEQ ID NO:2; the C-telopeptide is from amino acid residue 1067 to amino acid residue 1091 of SEQ ID NO:2; and the C-propeptide is from amino acid residue 1092 to amino acid residue 1336 of SEQ ID NO:2. In one embodiment, the collagen starting material for use in the present methods comprises a collagen having an amino acid sequence of from amino acid residue 38 to amino acid residue 1066 of SEQ ID NO:2. In another embodiment, the collagen starting material for use in the present methods has an amino acid sequence of from amino acid residue 38 to amino acid residue 1066 of SEQ ID NO:2.

Other collagens can be used as the collagen starting material for producing double-crosslinked collagen materials according to the methods of the present invention. In one embodiment, the collagen starting material for use in the present methods is a collagen having an amino acid sequence of SEQ ID NO:2, wherein the ammo acid sequence contains an isoleucine to proline substitution at amino acid residue 822 of SEQ ID NO:2. In another embodiment, the collagen starting material for use in the present methods is a collagen having an amino acid sequence of from amino acid residue 38 to amino acid residue 1066 of SEQ ID NO:2, wherein the amino acid sequence contains an isoleucine to proline substitution at amino acid residue 822 of SEQ ID NO:2 (collagen type III-A).

In another embodiment, the collagen starting material for use in the present methods is a collagen having an amino acid sequence of SEQ ID NO:2, wherein the amino acid sequence contains proline substitutions at amino acid residues 817, 820, 823, 826, and 829 of SEQ ID NO:2. In another embodiment, the collagen starting material for use in the present methods is a collagen having an amino acid sequence of from amino acid residue 38 to amino acid residue 1066 of SEQ ID NO:2, wherein the amino acid sequence contains proline substitutions at amino acid residues 817, 820, 823, 826, and 829 of SEQ ID NO:2 (collagen type III-B).

In yet another embodiment, the collagen starting material for use in the present methods is a collagen having an amino acid sequence of SEQ ID NO:2, wherein the amino acid sequence contains proline substitutions at amino acid residues 817, 820, 822, 823, 826, and 829 of SEQ ID NO:2. In another embodiment, the collagen starting material for use in the present methods is a collagen having an amino acid sequence of from amino acid residue 38 to amino acid residue 1066 of SEQ ID NO:2, wherein the amino acid sequence contains proline substitutions at amino acid residues 817, 820, 822, 823, 826, and 829 of SEQ ID NO:2 (collagen type III-C).

In yet another embodiment, the collagen starting material for use in the present methods is a collagen having an amino acid sequence of SEQ ID NO:2, wherein the amino acid sequence contains proline substitutions at amino acid residues 265, 300, 402, 414, 468, 471, 543, 567, 576, 603, 618, 693, 717, 738, and 900 of SEQ ID NO:2. In another embodiment, the collagen starting material for use in the present methods is a collagen having an amino acid sequence of from amino acid residue 38 to amino acid residue 1066 of SEQ ID NO:2, wherein the amino acid sequence contains proline substitutions at amino acid residues 265, 300, 402, 414, 468, 471, 543, 567, 576, 603, 618, 693, 717, 738, and 900 of SEQ ID NO:2 (collagen type III-D).

In certain embodiments of the present invention, the collagen starting material used for producing double-crosslinked collagen materials is a collagen free of intramolecular crosslinks, free of intermolecular crosslinks, free of endogenous crosslinks, free of propeptide sequence, free of telopeptide sequence (i.e., atelopeptide collagen), or free of hydroxylation, including, for example, free of proline hydroxylation. In particular embodiments, the collagen starting material used for producing double-crosslinked collagen materials is a recombinant collagen, including a recombinant human collagen, wherein the recombinant collagen or recombinant human collagen is free of intramolecular crosslinks, free of intermolecular crosslinks, free of endogenous crosslinks, free of propeptide sequence, free of telopeptide sequence, or free of hydroxylation, including free of proline hydroxylation.

Production of other collagens suitable for use in the present compositions and methods can be specifically engineered using molecular biology techniques know to one of skill in the art. Such collagens can be modified by, e.g., an alteration in the polypeptide coding sequence, including deletion, substitutions, insertions, etc., to increase resistance to degradation. For example, recombinant collagens with alterations in the amino acid sequence at specific protease cleavage sites can be produced. Accordingly, in one embodiment, the present invention provides novel compositions comprising collagen, wherein the collagen is a recombinant Type III collagen. In one aspect, the recombinant Type III collagen comprises amino acid residue 38 to amino acid residue 1066 of SEQ ID NO:2, wherein the amino acid sequence contains proline substitutions at amino acid residues 817, 820, 823, 826, and 829 of SEQ ID NO:2. In another aspect, the recombinant Type III collagen comprises amino acid residue 38 to amino acid residue 1066 of SEQ ID NO:2, wherein the amino acid sequence contains proline substitutions at amino acid residues 817, 820, 822, 823, 826, and 829 of SEQ ID NO:2. In yet another aspect, the recombinant Type III collagen comprises amino acid residue 38 to amino acid residue 1066 of SEQ ID NO:2, wherein the amino acid sequence contains proline substitutions at amino acid residues 265, 300, 402, 414, 468, 471, 543, 567, 576, 603, 618, 693, 717, 738, and 900 of SEQ ID NO:2.

The methods of the present invention are particularly useful for producing double-crosslinked collagen materials using recombinant collagen (e.g., recombinant human collagen) as the collagen starting material. Unlike naturally-derived collagens, recombinant collagens lack intermolecular and intramolecular crosslinks that, if present, help stabilize the collagen material (including collagen fibrils) under conditions suitable for various crosslinking reactions, including, for example, basic pH conditions (e.g., pH ≧8) or increased temperature (e.g., temperature ≧40° C.). Under such conditions, recombinant collagens and, in particular, recombinant collagen fibrils made from recombinant collagens, are unstable, resulting in fibril dissolution and triple helix melting. The present invention relates, in part, to the unexpected finding that by performing a first crosslinking reaction on recombinant collagen materials under conditions sufficient to maintain helix and fibril structure, such first crosslinking reaction provides crosslinking sufficient to stabilize the recombinant collagen to allow for subsequent crosslinking of the collagen by a second crosslinking agent under conditions that would otherwise result in fibril dissolution or helix melting.

Crosslinking Agents

In certain embodiments, the double-crosslinked collagen materials of the present invention are prepared by sequential crosslinking of collagen with a first crosslinking agent and a second crosslinking agent. In particular embodiments, the crosslinking agent for use in the present methods is a zero-length crosslinking agent or a homobifunctional crosslinking agent. It is a particular aspect of the present methods that the first crosslinking agent be different from the second crosslinking agent.

Crosslinking agents useful in the present methods include, for example, a carbodiimide, a bis-epoxide, or a homobifunctional aldehyde. In one aspect, the carbodiimide is 1-ethyl-3-(3-dimethylaminopropyl)-carbodiimide hydrochloride (EDC), 1-Cyclohexyl-3-(2-morpholinoethyl)carbodiimide (CMC), dicyclohexyl carbodiimide (DCC), or diisopropyl carbodiimide (DIC). In another aspect, the bis-epoxide is 1,4-butanediol diglycidyl ether (BDDE), 1,6-hexanediol diglycidyl ether (HDDGE), polyethylene glycol diglycidyl ether (PEGDE), polypropylene glycol diglycidyl ether (PPGDE), polytetramethylene glycol diglycidyl ether (PTMGDGE), neopentyl glycol diglycidyl ether (NPGDGE), polyglycerol polyglycidyl ether (PGPGE), diglycerol polyglycidyl ether (DGPGE), trimethylolpropane polyglycidyl ether (TMPPGE or EX-321), pentaerythritol polyglycidyl ether (PEPGE or EX-411), sorbitol polyglycidyl ether (SPGE or EX-614), sorbitan polyglycidyl ether, resorcinol diglycidyl ether (RDGE), glycerol polyglycidyl ether (GPGE or EX-313), glycerol triglycidyl ether (GTGE or EX-314), or ethylene glycol diglycidyl ether (EGDGE or EX-810). In yet another aspect, the homobifunctional aldehyde is formaldehyde (FA), glyoxal, malondialdehyde, succinaldehyde, glutaraldehyde (GA), or adipaldehyde. Further exemplary crosslinking agents useful for crosslinking collagen according to the present methods are described in U.S. Pat. Nos. 5,880,242 and 6,117,979 and in Zeeman et al., 2000, J Biomed Mater Res. 51(4):541-8, van Wachem et al., 2000, J Biomed Mater Res. 53(1):18-27, van Wachem et al., 1999, J Biomed Mater Res. 47(2):270-7, Zeeman et al., 1999, J Biomed Mater Res. 46(3):424-33, Zeeman et al., 1999, Biomaterials 20(10):921-31, each of which is hereby incorporated by reference herein in its entirety.

Although not intending to be bound by any particular theory of operation, it is understood in the art that the crosslink initiated by an aldehyde crosslinking agent, such as gluteraldehyde, occurs by the reaction of the aldehyde group of the crosslinking agent with collagen α-amine groups of either lysine or hydroxylysine residues, thus forming an amide crosslink. In such a crosslink, two amine (—NH₃) groups are used in every aldehyde-induced primary amine crosslink.

The crosslink initiated by a carbodiimide crosslinking agent occurs by the activation of the free carboxyl groups of glutamic acid and aspartic acid moieties in collagen. Activation of the carboxyl groups with a carbodiimide crosslinking agent, such as, for example, 1-ethyl-3-(3-dimethylaminopropyl)carbodiimide HCl (EDC), gives o-acylisourea groups. A condensation reaction by nucleophilic attack of a free α-amine group of either lysine or hydroxylysine residues with urea as a leaving group results in formation of an amide crosslink.

The crosslink initiated by an epoxide, such as, for example, BDDE, occurs at basic pH conditions (e.g., pH >8.0) by the activation of carboxyl groups and amine groups in collagen. The reaction of the epoxide functional groups with hydroxyls requires basic pH conditions (e.g., pH range of pH 11-12), while amine nucleophiles react at more moderate alkaline pH values (e.g., pH of at least pH 9). The reaction proceeds with hydrolysis of epoxy groups to form a β-hydroxy group that can be oxidized to create reactive aldehydes. These reactive aldehydes can then react with collagen α-amine groups of either lysine or hydroxylysine residues to form an amide crosslink.

When an epoxide compound is used as the first and/or second crosslinking agent, it is typical to quench any pendant epoxide groups that remain in the crosslinked collagen material. This quenching may be carried out, for example, by treating the crosslinked collagen material with an excess of glycine. This quenching step may be carried out after the first crosslinking reaction and before the second crosslinking reaction when an epoxide compound is used as the first crosslinking agent. Alternatively, the quenching step may be carried out after the second crosslinking reaction when an epoxide compound is used as the first and/or second crosslinking agent. Typically, the quenching step is carried out after the second crosslinking reaction.

The inventors have found that when basic pH (e.g. >pH 8.0) conditions are used for crosslinking, e.g. when using an epoxide such as BDDE, the high pH may encourage dissolution of the collagen, thereby reducing the yield of crosslinked collagen. To solve this problem, the present inventors have found that crosslinking the collagen with a first crosslinking agent under conditions that do not involve a basic pH before crosslinking with a second crosslinking agent at a basic pH improves the yield of crosslinked collagen. Without wishing to be bound by a particular theory, it is thought that the first crosslinking agent stabilizes the collagen and reduces dissolution of the collagen when the pH is raised to a basic level for the second crosslinking agent.

In one embodiment, the first crosslinking agent is a homobifunctional aldehyde compound, typically glutaraldehyde, and the second crosslinking agent is an epoxide compound, typically BDDE. The glutaraldehyde crosslinking reaction is typically carried out at a neutral pH, typically 7.2. The BDDE crosslinking reaction is typically carried out at a basic pH, e.g. 8, 9, 10, 11 or 12.

Characterization of Double-Crosslinked Collagen Materials

Double-crosslinked collagen materials of the present invention can be characterized using various methods available and known to one of skill in the art. For example, double-crosslinked collagen materials of the present invention are characterized by a determination of the extent or degree of crosslinking of the double-crosslinked collagen materials. The extent or degree of crosslinking of the double-crosslinked collagen materials is measured using a 2,4,6-trinitrobenzene sulfonic acid (TNBS) assay. TNBS is a rapid and sensitive reagent useful for the determination of free primary amino groups in protein materials. (Bubnis et al. (1992) Anal Biochem 207:129-133.) Primary amino groups, such as those found on lysine residues in collagen, form a chromogenic derivative (TNP-lysine) upon reaction with TNBS, which can be measured for optical density in a spectrophotometer. (Everaerts et al. (2004) Biomaterials 25:5523-5530.)

In an exemplary TNBS assay, a collagen sample (e.g., a double-crosslinked collagen material) is suspended in 1 ml of 0.1M sodium carbonate, pH 9.0. To the collagen suspension, 1 ml of 0.5% TNBS is added and the collagen/TNBS solution is allowed to react for 2 hours at 40° C. The collagen solution is then solubilized with 3 ml of 6 N HCl and incubated for 1.5 hours at 60° C. Following incubation, the solubilized collagen solution is diluted with 5 ml deionized water, resulting in a 10 ml solution. Of that 10 ml solution, half (5 ml) is extracted with 3×10 ml of ethyl ether. The remaining aqueous solution is diluted to 20 ml final volume and measured for optical density at 345 nm (OD₃₄₅) in a spectrophotometer (Molecular Devices, Sunnyvale Calif.). The amount of free lysine in the collagen sample is determined from the absorbance reading using equations previously described. (Everaerts et al. (2004) Biomaterials 25:5523-5530.) In this TNBS assay, the amount of free lysine in the collagen sample is used to measure the extent of crosslinking; a lower amount of free lysine is indicative of a greater extent of crosslinking in the collagen sample.

The double-crosslinked collagen materials of the present invention can have a high degree of crosslinking which can be determined as described above. In certain embodiments, the extent or degree of crosslinking of the double-crosslinked collagen materials is about 40-100%, about 45-100%, about 50-100%, about 55-100%, about 60-100%, about 65-100%, about 70-100%, about 75-100%, about 80-100%, about 85-100%, or about 80-90%. In other embodiments, the extent or degree of crosslinking of the double-crosslinked collagen materials is about 50%, about 55%, about 60%, about 65%, about 70%, about 75%, about 80%, about 85%, about 90%, or about 95%. In particular embodiments, the extent or degree of crosslinking of the double-crosslinked collagen materials of the present invention is greater than 69%.

Double-crosslinked collagen materials of the present invention can also be characterized by thermal stability. Determination of thermal stability can be used to determine crosslinking efficiency (Petite et al. (1990) J Biomed Mater Res 24:179-87) and to predict in vivo persistence (DeLustro et al. (1986) J Biomed Mater Res 20:109-20) of double-crosslinked collagen materials. Thermal stability of the double-crosslinked collagen materials produced using methods of the present invention can be determined using any method known to one skilled in the art, including, for example, by differential scanning calorimetry (DSC). (See, e.g., Sionkowska (2005) J Photochem Photobiol B 80:87-92.) DSC is a thermoanalytical technique that can be used to determine the melting temperature (Tₘ) of a collagen material by recording the heat required for the collagen sample to undergo a phase transition from collagen to gelatin. Melting temperature correlates with thermal stability.

In an exemplary assay, DSC measurements are performed with 200 μl samples of various formulations of double-crosslinked collagen material solutions (30-40 mg/ml) in 20 mM sodium phosphate (pH 7.2). Thermograms are recorded from 10° C. to 80° C. with a scan rate of 1.00° C./minute in a DSC (Mettler Toledo, Model DSC822, Columbus Ohio). Deconvolution of peaks in the DSC thermograms are performed with STAR software (Mettler Toledo, Columbus Ohio) to calculate the melting temperature of the double-crosslinked collagen material.

The double-crosslinked collagen materials of the present invention are thermally stable. In certain embodiments, the melting temperature of the double-crosslinked collagen material of the present invention is 60° C. or greater, about 60-80° C., about 65-80° C., about 70-80° C., or about 75-80° C. In other embodiments, the melting temperature of the double-crosslinked collagen material of the present invention is about 60° C., about 65° C., about 70° C., about 75° C., or about 80° C. In particular embodiments, the melting temperature of the double-crosslinked collagen material of the present invention is about 75° C.

The double-crosslinked collagen materials of the present invention can also be characterized by determining the amount of pendant epoxy groups in the double-crosslinked material. Active pendant epoxy groups may be introduced into the collagen molecules after crosslinking with epoxide crosslinking agents (e.g., BDDE). In the context of tissue implantation, these active pendant epoxy groups have the potential to react with surrounding tissue following implantation. (See Zeeman et al. (1999) Biomaterials 20:921-931.) Therefore, in certain embodiments, it may be useful to determine the content of the pendant epoxy groups in the double-crosslinked collagen material.

Pendant epoxy group content of the double-crosslinked collagen materials produced using methods of the present invention may be determined using any method known to one skilled in the art. For example, the pendant epoxy group content of a double-crosslinked collagen material produced using methods of the invention is measured by a pendant epoxy group assay. (Zeeman et al. (2000) J Biomed Mater Res 51:541-854.) In this assay, the “starting” amine content of a double-crosslinked collagen material is determined by TNBS assay (see above). Next, the double-crosslinked collagen material is treated with lysine methyl ester dihydrochloride (LM). Following treatment with LM, the “final” amine content of the double-crosslinked collagen material is determined by TNBS assay. The pendant epoxy content is equal to the “final” amine content of the double-crosslinked collagen material (after LM treatment) minus the “starting” free amine content (before the LM addition) of the double-crosslinked collagen material.

In certain embodiments, the active pendant epoxy group content of the double-crosslinked collagen material of the present invention is about 0.01-2.5 moles, about 0.5-2.5 moles, about 0.75-2.5 moles, about 1.0-2.5 moles, about 1.25-2.5 moles, about 1.5-2.5 moles, or about 2-2.5 moles. In other embodiments, the active pendant epoxy group content of the double-crosslinked collagen material of the present invention is about 0.01 moles, about 0.5 moles, about 0.75 moles, about 1.0 mole, about 1.5 moles, about 2.0 moles, or about 2.5 moles. In particular embodiments, the active pendant epoxy group content of the double-crosslinked collagen material of the present invention is about 0.8 moles.

In vitro persistence of collagen materials may be used to predict in vivo persistence following implantation. In vitro persistence of double-crosslinked collagen materials produced using methods of the present invention may be determined using any method known to one skilled in the art. For example, in vitro persistence of a double-crosslinked collagen material produced using methods of the present invention is determined by a collagenase digestion assay. (McPherson et al. (1986) Journal of Biomedical Material Research 20:79-92.) In an exemplary assay, approximately 4 mg of double-crosslinked collagen material is suspended in 0.5 ml of a bacterial collagenase (Collagenase Form III, Advanced Biofactures Corp., Lynbrook N.Y.) solution (95 U), corresponding to a ratio of approximately 24 U bacterial collagenase per milligram of collagen materials. The mixture is incubated at 37° C. with samples being taken from the mixture at 24 hours and 1 week. Samples taken from each vial are centrifuged and 100 μl of the resulting supernatant is measured for optical density at 225 nm (OD₂₂₅) in a spectrophotometer (Molecular Devices, Sunnyvale Calif.). Absorbance serves as an index of the digested, soluble collagen where an increase in absorbance indicates increased collagen digestion and decreased persistence in vitro.

Persistence is a desirable feature of any material to be implanted and used in various tissue augmentation procedures. In vivo persistence of the double-crosslinked collagen materials produced using methods of the present invention may be determined using any method known to one skilled in the art. In vivo persistence of double-crosslinked collagen materials produced using methods of the present invention is determined using a rodent model of in vivo persistence. In this model, collagen materials are implanted in a rodent by subcutaneous injection. The persistence of the collagen material implants are then evaluated by determining the number and/or wet weight of the original implants that are present at the injection sites at various time points post-implantation. In an exemplary model, male Wistar rats (Charles River Laboratories, Wilmington Mass.) are shaved and an 8 cm×6 cm site for injection is marked the day prior to implantation. Collagen material is resuspended in PBS to a final collagen concentration of 35 mg/ml. Implants are made by subcutaneous injection of 0.5 ml of the 35 mg/ml suspension of collagen in PBS on the dorsal flank. The collagen suspension is injected using a 1 cc syringe with a 30-gauge needle. Each animal receives four separate injections of collagen material. Animals are analyzed at 9 and 16 months post-implantation. Implants are surgically removed and dissected free from surrounding tissue, weighed, and examined macroscopically for appearance and texture. In vivo persistence of the collagen implants is evaluated by determining the number and wet weight of the original implants that are present at the injection sites at each of the time points.

In vivo persistence of double-crosslinked collagen materials produced using methods of the present invention can also be determined by evaluating the longevity of the collagen materials of the present invention following implantation, such as by visual or palpable assessment, for example, using Global Aesthetic Improvement Scale (GAIS) ratings, or by assessing in vitro resistance to metalloprotease degradation (see, e.g., Example 2), etc. GAIS is based on a physician's assessment of the overall improvement, e.g., cosmetic improvement, in a treated area, e.g., nasolabial fold, by comparing the patient's appearance after treatment to that before treatment. GAIS ratings include: very much improved (optimal cosmetic result for the implant in the patient); much improved (marked improvement in appearance from the initial condition, but not completely optimal for this patient); improved (obvious improvement in appearance from initial condition); no change (the appearance is essentially the same as the original condition); and worse (the appearance is worse than the original condition).

Commercial bovine collagen dermal fillers have been shown to cause hypersensitivity reactions in 1-3% of patients. (Moody et al. (2001) Dermatol Surg 27:789-91.) In addition, increased anti-bovine collagen antibody titers following implantation have been observed as well as reports of connective tissue disease arising after bovine collagen injections. (Frank et al. (1991) Plast Reconstr Surg 87:1080-8.) As a result of these adverse reactions, patients planning treatment with a bovine collagen product need to take an allergy test approximately 1 month prior to the expected procedure date to test for immunogenicity. Therefore, implantable collagen materials having decreased immunogenicity are desirable.

Immunogenicity of the double-crosslinked collagen materials produced using methods of the present invention may be determined using any method known to one skilled in the art. For example, immunogenicity of a double-crosslinked collagen material produced using methods of the present invention is determined using a rodent model of immunogenicity. (Quteish et al. (1991) J Periodontal Res 26:114-121.) In this model, double-crosslinked collagen materials are implanted in a rodent by subcutaneous injection. The immunogenicity of the double-crosslinked collagen materials are evaluated at various time points following implantation by determining the presence or absence of antibodies directed against the implanted collagen in the animal's serum. In an exemplary model, male Wistar rats (Charles River Laboratories, Wilmington Mass.) are shaved and an 8 cm×6 cm site for injection is marked the day prior to implantation. Collagen material is resuspended in PBS to a final collagen concentration of 35 mg/ml. Implants are made by subcutaneous injection of 0.5 ml of the 35 mg/ml suspension of collagen in PBS on the dorsal flank. The collagen suspension is injected using a 1 cc syringe with a 30-gauge needle. Each animal receives four separate injections of collagen material. Serum samples are taken from each animal at 7, 9, and 16 months post implantation. Serum samples are analyzed for the presence of antibodies directed against the collagen type(s) present in the implanted material by standard sandwich ELISA techniques. (Quteish et al. (1991) J Periodontal Res 26:114-121.) Immunogenicity of the collagen implants are determined by the antibody titer levels of antibodies directed against the implanted collagen at each time point. The absence of antibodies directed against the implanted collagen material in this assay indicates that the collagen material is non-immunogenic.

Double-Crosslinked Collagen Materials

The present invention provides double-crosslinked collagen materials useful, for example, for augmenting or replacing tissue of a mammal. In certain embodiments, the double-crosslinked collagen materials of the invention have advantageous manipulability, extrudability, and intrudability properties.

The present invention relates, in part, to the discovery that double-crosslinked recombinant collagen displays greater persistence than does single-crosslinked or non-crosslinked recombinant collagen. In particular, the present invention demonstrates that implantable double-crosslinked recombinant collagen materials have persistence greater than that of implantable single-crosslinked or non-crosslinked recombinant collagen materials, e.g., double-crosslinked recombinant collagen will persist longer and degrade at a slower rate than single-crosslinked or non-crosslinked recombinant collagen. Therefore, in one embodiment, the present invention provides an implantable composition comprising double-crosslinked recombinant collagen. In other embodiments, the present invention provides implantable compositions that comprise collagen, wherein the collagen comprises a specific arid predetermined amount of double-crosslinked recombinant collagen, sufficient to give increased persistence to the final product. In a particular embodiment, the double-crosslinked recombinant collagen is double-crosslinked recombinant human collagen.

In one embodiment, the invention provides double-crosslinked recombinant collagen suitable for implantation into a human or animal body. Such a double-crosslinked recombinant collagen implant is suitable for medical or cosmetic use. Typically, double-crosslinked recombinant collagen according to the invention is implanted or injected into various regions of the skin or dermis, depending on the particular application or cosmetic procedure, including dermal, intradermal, and subcutaneous injection or implantation. The double-crosslinked collagen materials of the present invention can also be injected or implanted superficially, such as, for example, within the papillary layer of the dermis, or can be injected or implanted within the reticular layer of the dermis. Materials for injection or implantation into the skin, in particular for cosmetic benefit, are often referred to in the art as “dermal fillers”. Accordingly, in one embodiment, a dermal filler, typically a cosmetic dermal filler, comprising double-crosslinked recombinant collagen according to the invention is provided.

In various embodiments, the present invention encompasses double-crosslinked collagen materials comprising a collagen, wherein the collagen is prepared according the methods of the present invention

Formulations of Double-Crosslinked Collagen Materials

The double-crosslinked collagen materials of the present invention may be used to produce implantable collagen compositions. Production of implantable collagen compositions has been described in, e.g., International Publication No. WO 2006/052451, the contents of which is hereby incorporated by reference herein in its entirety. In certain embodiments, the present invention provides implantable collagen compositions, comprising at least one double-crosslinked collagen material. The double-crosslinked collagen material can be any double-crosslinked collagen of the invention, for instance double-crosslinked “fibril forming” collagen materials prepared by one of the methods described herein. In one aspect, the implantable collagen composition comprises double-crosslinked recombinant type III collagen material.

The double-crosslinked collagen materials of the present invention can be formulated or used at any concentration useful to those of skill in the art. In certain embodiments, the formulations of the materials of the invention comprise 0.1-100 mg/ml, 1-100 mg/ml, 1-75 mg/ml, 1-50 mg/ml, 1-40 mg/ml, 10-40 mg/ml or 20-40 mg/ml collagen. In other embodiments, the formulations of the materials of the invention comprise about 5 mg/ml, 10 mg/ml, 15 mg/ml, 20 mg/ml, 25 mg/ml, 30 mg/ml, 35 mg/ml, 40 mg/ml, 45 mg/ml or 50 mg/ml collagen. In a particular embodiment, the present invention provides formulations of double-crosslinked collagen materials comprising about 35 mg/ml collagen.

It is understood that the compositions of the present invention can include additional components suitable to the particular formulation. For example, in certain embodiments, the implantable compositions of the present invention are intended for injection and are formulated in aqueous solutions. The compositions can be formulated to include pharmaceutically acceptable carriers and excipients. Such carriers and excipients are well-known in the art and can include, e.g., water, phosphate buffered saline (PBS) solutions, various solvents, and salts, etc., for example, physiologically compatible buffers including physiological saline buffers such as Hanks solution and Ringer's solution.

The amount of double-crosslinked collagen material appropriately included in a particular formulation is determined as standard in the art for such formulations, and is dictated by the intended use. In certain embodiments, the present invention provides implantable compositions comprising double-crosslinked collagen material wherein the collagen material is in aqueous solution at a concentration between about 20 to about 120 mg/ml. In some embodiments, the double-crosslinked collagen material is in aqueous solution at a concentration between about 30 to about 90 mg/ml; or a concentration of between about 20 to 65 mg/ml; or a concentration of between about 25 to 40 mg/ml. In particular embodiments, the double-crosslinked collagen materials of the present invention have a collagen material concentration'of about 35 mg/ml or a collagen material concentration of about 65 mg/ml.

Methods of Using Double-Crosslinked Collagen Materials

The double-crosslinked collagen materials provided herein can be used in any method known or contemplated by those skilled in the art. In particular, the present double-crosslinked collagen materials can be used in any of the numerous medical and cosmetic applications, including tissue augmentation procedures, in which collagen is currently used and in which compositions containing double-crosslinked collagen materials and having greater persistence, improved handling, and/or less variability may be desired. The present double-crosslinked collagen materials are suitable for use in tissue augmentation procedures. Use of the present double-crosslinked collagen materials in cosmetic as well as in medical procedures is specifically provided.

In one aspect, the present invention provides implantable compositions containing double-crosslinked collagen materials suitable for use in soft tissue augmentation procedures. The present compositions can be implanted or injected into various regions of the skin or dermis, depending on the particular application or cosmetic procedure, including dermal, intradermal, and subcutaneous injection or implantation. The double-crosslinked collagen materials of the present invention can also be injected or implanted superficially, such as, for example, within the papillary layer of the dermis, or can be injected or implanted within the reticular layer of the dermis.

In addition to soft tissue augmentation, use of the double-crosslinked collagen materials for hard tissue augmentation is provided by the present invention. The present double-crosslinked collagen materials are useful in various hard tissue augmentation applications, including, for example, as a bone-void filler, dental implant, etc.

Cosmetic uses of the double-crosslinked collagen materials of the present invention include treatment of fine lines, such as fine superficial facial lines, wrinkles, and scars, as well as treatment of pronounced lines, wrinkles, and scars. In some aspects, the double-crosslinked collagen materials of the present invention are used for other cosmetic uses, including treatment for or reducing transverse forehead lines, glabellar frown lines, nasolabial fold, vermilion border, periorbital lines, vertical lip lines, oral commissure, etc., as well as defining the lip border. The double-crosslinked collagen materials of the present invention are also useful for correcting contour deformities and distensible acne scars, or for treating other tissue defects, such as, for example, atrophy from disease or trauma or surgically-induced irregularities.

In certain embodiments, the double-crosslinked collagen materials of the present invention are used for surgical procedures involving tissue augmentation, tissue repair, or drug delivery. In some aspects, the double-crosslinked collagen materials are used for tissue augmentation in conditions such as urinary incontinence, vasicoureteral reflux, and gastroesophageal reflux. For example, double-crosslinked collagen materials of the present invention may be used to add tissue bulk to sphincters, such as a gastric or urinary sphincter, to provide proper closure and control. In instances of urinary incontinence, such as stress incontinence in women or incontinence following a prostatectomy in men, the double-crosslinked collagen materials of the invention may be provided to further compress the urethra to assist the sphincter muscle in closing, thus avoiding leakage of urine from the bladder.

Similarly, gastroesophageal reflux disease (GERD, also known as peptic esophagitis and reflux esophagitis) is a disorder that affects the lower esophageal sphincter, the muscle connecting the esophagus with the stomach. GERD occurs when the lower esophageal sphincter is incompetent, weak, or relaxes inappropriately, allowing stomach contents to flow up into the esophagus (i.e., reflux). Malfunction of the lower esophageal sphincter muscles, such as that resulting from muscle tonal loss, can lead to incomplete closure of the lower esophageal sphincter, causing back up of acid and other contents from the stomach into the esophagus. Poor response to dietary modification or medical treatment may require surgery to correct the dysfunction. In one embodiment, double-crosslinked collagen materials of the present invention are used in such procedures and, for example, are injected into the area of the esophageal sphincter to provide bulk to the lower esophageal sphincter.

In other embodiments, the double-crosslinked collagen materials of the invention are used to fill or block voids and lumens within the body. Such voids may include, but are not limited to, various lesions, fissures, diverticulae, cysts, fistulae, aneurysms, or other undesirable voids that may exist within the body; and lumens may include, but are not limited to, arteries, veins, intestines, Fallopian tubes, and trachea. For example, an effective amount of the present material may be administered into the lumen or void to provide partial or complete closure, or to facilitate repair of damaged tissue.

In other aspects, tissue repair is achieved by providing the double-crosslinked collagen material of the present invention to an area of tissue that has been diseased, wounded, or removed. In some embodiments, double-crosslinked collagen materials of the invention are used to fill in and/or smooth out soft tissue defects such as pockmarks or scars. In such cases, a formulation of the present invention is injected beneath the imperfection. The improved persistence of the present double-crosslinked collagen materials would be beneficial, e.g., by reducing the number and frequency of treatments required to obtain a satisfactorily result. In certain embodiments, the double-crosslinked collagen materials are used for intracordal injections of the larynx, thus changing the shape of this soft tissue mass and facilitating vocal function. Such use is specifically provided for the treatment of unilateral vocal cord paralysis. Further, the present invention provides use of the double-crosslinked collagen materials in mammary implants, or to correct congenital anomalies, acquired defects, or cosmetic defects.

The present double-crosslinked collagen materials can also be used in various surgical or other procedures for remodeling or restructuring of various external or internal features, e.g., plastic surgery for corrective or cosmetic means, etc.

In any of the embodiments described above, the present double-crosslinked collagen materials may be used for drug delivery, for example, to deliver drugs to an injection site. The drugs can be delivered in a sustained manner from an in vivo depot formed by the double-crosslinked collagen upon injection of an implantable composition of the present invention. Drugs delivered in this manner may thus enhance tissue repair, and could provide additional therapeutic benefit.

In additional embodiments, the invention further contemplates incorporation of cells into the double-crosslinked collagen materials to provide a means for delivering cells to repopulate a damaged or diseased tissue or to provide products synthesized by the cells to the tissues surrounding the injection site.

In any of the embodiments described above, the double-crosslinked collagen materials of the present invention may be delivered or administered by any suitable method known or contemplated by those of skill in the art. The invention specifically contemplates delivery by injection, e.g., using a syringe. In some embodiments, the double-crosslinked collagen materials may additionally contain a biocompatible fluid that functions as a lubricant to improve the injectability of the formulation. The double-crosslinked collagen materials of the invention can be introduced into the tissue site by injection, including, e.g., intradermal, subdermal, or subcutaneous injection.

Kits Comprising Double-Crosslinked Collagen Materials

One embodiment of the present invention provides kits comprising the double-crosslinked collagen materials of the invention. For example, the present invention provides kits for augmenting or replacing tissue of a mammal. The kits comprise one or more double-crosslinked collagen materials of the present invention in a package for distribution to a practitioner of skill in the art. The kits can comprise a label or labeling with instructions on using the double-crosslinked collagen material for augmenting or replacing tissue of a mammal according to the methods of the invention. In certain embodiments, the kits can comprise components useful for carrying out the methods such as means for administering a double-crosslinked collagen material such as one or more syringes, canulas, catheters, needles, etc. In certain embodiments, the kits can comprise components useful for the safe disposal of means for administering the double-crosslinked collagen material (e.g. a ‘sharps’ container for used syringes). In certain embodiments, the kits can comprise double-crosslinked collagen material in pre-filled syringes, unit-dose or unit-of-use packages.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18^{th} ed., Mack Publishing Co.; Hardman, J. G., Limbird, L. E., and Gilman, A. G., eds. (2001) The Pharmacological Basis of Therapeutics, 10^{th} ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2^{nd} ed., Springer Verlag.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference. Nothing herein is to be construed as an admission that the invention is not entitled to antedate any art described herein by virtue of prior invention.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Production of Double-Crosslinked Collagen

Double-crosslinked collagen was produced as follows. Various types of recombinant human collagens were prepared using methods previously described (see e.g., WO 2006/052451 and WO 1993/007889), and diluted with 10 mM HCl to 3.0 mg/ml to form a bulk collagen solution. A 200 ml sample of each collagen solution was mixed with 20 ml of fibrillogenesis buffer (0.2 M NaPO₄, pH 11.2). Fibrillogenesis (i.e., collagen fibril formation) occurred overnight or for six hours at room temperature.

Following collagen fibril formation, the collagen solution was centrifuged, the supernatant discarded, and the resulting pelleted collagen fibrils were resuspended in 20 mM NaPO₄ at pH 7.2 to form a 3.0 mg/ml collagen fibril solution. Next, the collagen in the collagen fibril solution was subjected to a first crosslinking reaction to produce a single-crosslinked collagen as follows. Various first crosslinking agents were added to the collagen fibril solutions produced as described above. For each first crosslinking reaction, several combinations of temperature, pH, and crosslinking reaction times were examined using various concentrations of the first crosslinking agents (see Table 1 below). Crosslinking agents used as first crosslinking agents in these studies are shown below in Table 1, and included: glutaraldehyde (GA); formaldehyde (FA); 1,4-butanediol diglycidyl ether (BDDE); and 1-ethyl-3-(3-dimethylaminopropyl)-carbodiimide hydrochloride (EDC).

Following the overnight first crosslinking reaction, a second crosslinking reaction was performed on the single-crosslinked collagen to produce a double-crosslinked collagen. Various concentrations of a second crosslinking agent were added to the single-crosslinked collagen solutions, and several combinations of temperature, pH, and crosslinking reaction times were examined (see Table 1). Crosslinking agents used as second crosslinking agents in these studies are shown below in Table 1, and included: glutaraldehyde (GA); 1-ethyl-3-(3-dimethylaminopropyl)-carbodiimide hydrochloride (EDC); 1,4-butanediol diglycidyl ether (BDDE); glycerol polyglycidyl ether (GPGE or EX-313); glycerol triglycidyl ether (GTGE or EX-314); ethylene glycol diglycidyl ether (EGDGE or EX-810); polypropylene glycol diglycidyl ether (PPGDE); neopentyl glycol diglycidyl ether (NPGDGE); trimethylolpropane polyglycidyl ether (TMPPGE or EX-321); and polyethylene glycol diglycidyl ether (PEGDE). At the end of the second crosslinking reaction, the double-crosslinked collagen solutions were centrifuged and the supernatant discarded. The resulting double-crosslinked collagen pellets were washed three times with water or a 20 mM sodium phosphate buffer (pH 7.2).

TABLE 1

| | | First Crosslinking | | | | | Second Crosslinking | | | | |
|---|---|---|---|---|---|---|---|---|---|---|---|
| | Recombinant | Crosslinking | | | | | Crosslinking | | | | |
| Material | Collagen Type | Agent | Conc., % | pH | Temp, ° C. | Time, hours | Agent | Conc. % | pH | Temp, ° C. | Time, hours |
| A | I | GA | 0.0012 | 7.2 | RT | 16 | BDDE | 4.0 | 10 | 30 | 16 |
| B | II | GA | 0.0012 | 7.2 | RT | 16 | BDDE | 4.0 | 10 | 30 | 16 |
| C | III | EDC | 0.15 | 7.2 | RT | 16 | BDDE | 4.0 | 10 | 30 | 16 |
| D | III | EDC | 0.5 | 7.2 | RT | 16 | BDDE | 4.0 | 10 | 30 | 16 |
| E | III | BDDE | 4.0 | 2.0 | 30 | 16 | — | — | — | — | — |
| F | III | BDDE | 4.0 | 4.5 | 30 | 16 | — | — | — | — | — |
| G | III | BDDE | 4.0 | 7.2 | 30 | 16 | — | — | — | — | — |
| H | III | BDDE | 4.0 | 10 | 30 | 16 | — | — | — | — | — |
| I | III | GA | 0.0035 | 7.2 | RT | 16 | GPGE | 4.0 | 10 | 30 | 16 |
| J | III | GA | 0.0035 | 7.2 | RT | 16 | GPGE | 4.0 | 10 | 30 | 40 |
| K | III | GA | 0.0035 | 7.2 | RT | 16 | GTGE | 4.0 | 10 | 30 | 16 |
| L | III | GA | 0.0035 | 7.2 | RT | 16 | GTGE | 4.0 | 10 | 30 | 40 |
| M | III | GA | 0.0035 | 7.2 | RT | 16 | EGDGE | 4.0 | 10 | 30 | 16 |
| N | III | GA | 0.0035 | 7.2 | RT | 16 | EGDGE | 4.0 | 10 | 30 | 40 |
| O | III | GA | 0.0035 | 7.2 | RT | 16 | — | — | — | — | — |
| P | I | FA | 0.0012 | 7.2 | RT | 16 | — | — | — | — | — |
| Q | I | FA | 0.0035 | 7.2 | RT | 16 | — | — | — | — | — |
| R | I | FA | 0.012 | 7.2 | RT | 16 | — | — | — | — | — |
| S | I | FA | 0.035 | 7.2 | RT | 16 | — | — | — | — | — |
| T | I | FA | 0.12 | 7.2 | RT | 16 | — | — | — | — | — |
| U | I | FA | 0.35 | 7.2 | RT | 16 | — | — | — | — | — |
| V | I | FA | 0.0012 | 7.2 | RT | 16 | BDDE | 4.0 | 10 | 30 | 16 |
| W | I | FA | 0.0035 | 7.2 | RT | 16 | BDDE | 4.0 | 10 | 30 | 16 |
| X | I | FA | 0.012 | 7.2 | RT | 16 | BDDE | 4.0 | 10 | 30 | 16 |
| Y | I | GA | 0.0012 | 7.2 | RT | 16 | PPGDE | 4.0 | 10 | 30 | 16 |
| Z | I | GA | 0.0012 | 7.2 | RT | 16 | EGDGE | 4.0 | 10 | 30 | 16 |
| AA | I | GA | 0.0012 | 7.2 | RT | 16 | NPGDGE | 4.0 | 10 | 30 | 16 |
| AB | I | GA | 0.0012 | 7.2 | RT | 16 | TMPPGE | 4.0 | 10 | 30 | 16 |
| AC | I | GA | 0.0012 | 7.2 | RT | 16 | PEGDE | 4.0 | 10 | 30 | 16 |
| AD | III | GA | 0.0004 | 10 | RT | 16 | — | — | — | — | — |
| AE | III | GA | 0.0012 | 10 | RT | 16 | — | — | — | — | — |
| AF | III | GA | 0.0036 | 10 | RT | 16 | — | — | — | — | — |
| AG | III | GA | 0.012 | 10 | RT | 16 | — | — | — | — | — |
| AH | III | GA | 0.036 | 10 | RT | 16 | — | — | — | — | — |
| AI | III | GA | 0.0035 | 7.2 | RT | 16 | EDC | 0.15 | 7.2 | RT | 16 |
| AJ | III | GA | 0.0035 | 7.2 | RT | 16 | EDC | 0.5 | 7.2 | RT | 16 |
| AK | III | EDC | 0.15 | 7.2 | RT | 16 | GA | 0.0035 | 7.2 | RT | 16 |
| AL | III | EDC | 0.5 | 7.2 | RT | 16 | GA | 0.0035 | 7.2 | RT | 16 |
| AM | III | EDC | 0.15 | 7.2 | RT | 16 | — | — | — | — | — |
| AN | III | EDC | 0.5 | 7.2 | RT | 16 | — | — | — | — | — |
| AO | III | EDC | 0.15 | 7.2 | RT | 16 | BDDE | 4.0 | 10 | 30 | 16 |
| AP | III | EDC | 0.5 | 7.2 | RT | 16 | BDDE | 4.0 | 10 | 30 | 16 |
| AQ | I | EDC | 0.15 | 7.2 | RT | 16 | BDDE | 4.0 | 10 | 30 | 16 |
| AR | I | EDC | 0.5 | 7.2 | RT | 16 | BDDE | 4.0 | 10 | 30 | 16 |
| AS | III | GA | 0.0012 | 7.2 | RT | 16 | BDDE | 4.0 | 10 | 30 | 16 |
| AT | III | GA | 0.0035 | 7.2 | RT | 16 | BDDE | 4.0 | 10 | 40 | 16 |
| AU | III | GA | 0.0012 | 7.2 | RT | 16 | BDDE | 2.5 | 10 | 40 | 16 |
| AV | III | GA | 0.0006 | 7.2 | RT | 16 | BDDE | 2.5 | 10 | 40 | 16 |
| AW | III | — | — | — | — | — | — | — | — | — | — |
| AX | III | GA | 0.0012 | 7.2 | RT | 16 | — | — | — | — | — |
| AY | III | GA | 0.0035 | 7.2 | RT | 16 | — | — | — | — | — |
| AZ | III | BDDE | 4.0 | 9.0 | 30 | 4 | — | — | — | — | — |
| BA | III | BDDE | 4.0 | 9.0 | 30 | 16 | — | — | — | — | — |
| BB | III | GA | 0.0012 | 7.2 | RT | 16 | BDDE | 2.0 | 9 | RT | 16 |
| BC | III | GA | 0.0012 | 7.2 | RT | 16 | BDDE | 2.0 | 9 | RT | 40 |
| BD | III | GA | 0.0012 | 7.2 | RT | 16 | BDDE | 2.0 | 9 | 30 | 16 |
| BE | III | GA | 0.0012 | 7.2 | RT | 16 | BDDE | 2.0 | 9 | 30 | 40 |
| BF | III | GA | 0.0012 | 7.2 | RT | 16 | BDDE | 4.0 | 9 | RT | 16 |
| BG | III | GA | 0.0012 | 7.2 | RT | 16 | BDDE | 4.0 | 9 | RT | 40 |
| BH | III | GA | 0.0012 | 7.2 | RT | 16 | BDDE | 4.0 | 9 | 30 | 16 |
| BI | III | GA | 0.0012 | 7.2 | RT | 16 | BDDE | 4.0 | 9 | 30 | 40 |
| BJ | III | GA | 0.0012 | 7.2 | RT | 16 | BDDE | 4.0 | 10 | 30 | 4 |
| BK | III | GA | 0.0012 | 7.2 | RT | 16 | BDDE | 4.0 | 10 | 30 | 16 |
| BL | III | GA | 0.0012 | 7.2 | RT | 16 | BDDE | 4.0 | 10 | 40 | 4 |
| BM | III | GA | 0.0012 | 7.2 | RT | 16 | BDDE | 4.0 | 10 | 40 | 16 |
| BN | III | GA | 0.0035 | 7.2 | RT | 16 | BDDE | 2.0 | 9 | RT | 16 |
| BO | III | GA | 0.0035 | 7.2 | RT | 16 | BDDE | 2.0 | 9 | RT | 40 |
| BP | III | GA | 0.0035 | 7.2 | RT | 16 | BDDE | 2.0 | 9 | 30 | 16 |
| BQ | III | GA | 0.0035 | 7.2 | RT | 16 | BDDE | 2.0 | 9 | 30 | 40 |
| BR | III | GA | 0.0035 | 7.2 | RT | 16 | BDDE | 2.0 | 10 | 30 | 4 |
| BS | III | GA | 0.0035 | 7.2 | RT | 16 | BDDE | 2.0 | 10 | 30 | 16 |
| BT | III | GA | 0.0035 | 7.2 | RT | 16 | BDDE | 2.0 | 10 | 40 | 4 |
| BU | III | GA | 0.0035 | 7.2 | RT | 16 | BDDE | 2.0 | 10 | 40 | 16 |
| BV | III | GA | 0.0035 | 7.2 | RT | 16 | BDDE | 4.0 | 9 | RT | 16 |
| BW | III | GA | 0.0035 | 7.2 | RT | 16 | BDDE | 4.0 | 9 | RT | 40 |
| BX | III | GA | 0.0035 | 7.2 | RT | 16 | BDDE | 4.0 | 9 | 30 | 4 |
| BY | III | GA | 0.0035 | 7.2 | RT | 16 | BDDE | 4.0 | 9 | 30 | 16 |
| BZ | III | GA | 0.0035 | 7.2 | RT | 16 | BDDE | 4.0 | 9 | 30 | 40 |
| CA | III | GA | 0.0035 | 7.2 | RT | 16 | BDDE | 4.0 | 9 | 40 | 4 |
| CB | III | GA | 0.0035 | 7.2 | RT | 16 | BDDE | 4.0 | 9 | 40 | 16 |
| CC | III | GA | 0.0035 | 7.2 | RT | 16 | BDDE | 4.0 | 10 | 30 | 4 |
| CD | III | GA | 0.0035 | 7.2 | RT | 16 | BDDE | 4.0 | 10 | 30 | 16 |
| CE | III | GA | 0.0035 | 7.2 | RT | 16 | BDDE | 4.0 | 10 | 40 | 4 |
| CF | III | GA | 0.0035 | 7.2 | RT | 16 | BDDE | 4.0 | 10 | 40 | 16 |

### Example 2

### Double Crosslinking Increased Yield of Collagen Material Versus Single Crosslinking

To examine the recovery yield of double-crosslinked collagen materials produced according to the methods of the present invention, the following studies were performed. Single-crosslinked or double-crosslinked collagen materials were produced as described above in Example 1 using the crosslinking agents shown below in Table 2. After the first and/or second crosslinking reaction, the concentration of single- and double-crosslinked collagen was determined for each set of reactions using a Bicinchoninic Acid (BCA) protein assay kit (Pierce, Rockford Ill.) according to the manufacturer's instructions. Recovery of collagen material (i.e., yield) was determined as the ratio of the amount of final collagen material collected to the amount of starting collagen material.

As shown in Table 2 below, methods of the present invention increased the yield of crosslinked collagen material recovered. For example, recombinant type III collagen crosslinked with a single crosslinking agent (GA or BDDE) resulted in a yield of 37%. However, recombinant type III collagen sequentially crosslinked with a first crosslinking agent (GA) and a second crosslinking agent (BDDE) resulted in yields of 78% to 90%. (See Table 2 below.) These results showed that the yield of collagen material crosslinked in a sequential manner using a first crosslinking agent and a second crosslinking agent (i.e., resulting in double-crosslinked collagen) was greater compared to the yield of collagen material crosslinked with a single crosslinking agent alone.

A deficiency of current collagen crosslinking methods, which is a major impediment to the production of commercially useful amounts of crosslinked collagen material, is the rapid dissolution of collagen fibrils at the higher pH required for effective crosslinking with epoxide crosslinking agents. These results suggested that methods of the present invention can reduce the dissolution of collagen fibrils during crosslinking reactions, thereby increasing yield of crosslinked collagen material. These results also suggested that sequential crosslinking of collagen using a first crosslinking agent and a second crosslinking agent may provide a useful means for obtaining more commercially viable yields of crosslinked collagen material.

TABLE 2

| Human | First Crosslinking | Second Crosslinking | Yield |
|---|---|---|---|
| Collagen Type | Agent (Concentration) | Agent (Concentration) | (%) |
| III | GA (12 ppm) | — | 37 |
| III | BDDE (4%) | — | 37 |
| III | GA (12 ppm) | BDDE (2%) | 90 |
| III | GA (35 ppm) | BDDE (1%) | 87 |
| III | GA (12 ppm) | BDDE (1%) | 85 |
| III | GA (6 ppm) | BDDE (4%) | 82 |
| III | GA (6 ppm) | BDDE (2%) | 82 |
| III | GA (6 ppm) | BDDE (1%) | 83 |
| III | GA (6 ppm) | BDDE (4%) | 85 |
| III | GA (6 ppm) | BDDE (2%) | 85 |
| III | GA (6 ppm) | BDDE (1%) | 86 |
| III | GA (3 ppm) | BDDE (4%) | 80 |
| III | GA (3 ppm) | BDDE (2%) | 80 |
| III | GA (3 ppm) | BDDE (1%) | 78 |
| I | GA (12 ppm) | PPGDE (4%) | 90 |
| I | GA (12 ppm) | EGDGE (4%) | 90 |
| I | GA (12 ppm) | NPGDGE (4%) | 100 |
| I | GA (12 ppm) | TMPPGE (4%) | 90 |
| I | GA (12 ppm) | PEGDE (4%) | 90 |
| I | EDC (0.15%) | BDDE (4%) | 100 |
| I | EDC (0.5%) | BDDE (4%) | 100 |

### Example 3

### Increased Thermal Stability of Double-Crosslinked Collagen

Thermal stability, as measured by differential scanning calorimetry (DSC), has been used to determine crosslinking efficiency (Petite et al. (1990) J Biomed Mater Res 24:179-87) and to predict in vivo persistence (DeLustro et al. (1986) J Biomed Mater Res 20:109-20) of crosslinked collagen materials. Thus, to examine the effect of double crosslinking methods of the present invention on the thermal stability of collagen material, the following studies were performed.

In this series of experiments, the effect of double crosslinking collagen on thermal stability of various types of collagen was examined by DSC. Various double-crosslinked recombinant human type III collagen materials were prepared as described above in Example 1 using different combinations of first and second crosslinking agents, as outlined in Table 3 below. Thermal stability of each collagen material was determined by measuring the melting temperature of the collagen materials using a differential scanning calorimeter (DSC). (Petite et al. (1990) J Biomed Mater Res 24:179-87). Briefly, DSC measurements were performed with 200 μl samples of various formulations of double-crosslinked collagen solutions (30-40 mg/ml) in 20 mM sodium phosphate (pH 7.2). Thermograms were recorded from a temperature range of 10° C. to 80° C. using a scan rate of 1.00° C./minute in a DSC (Mettler Toledo, Model DSC822, Columbus Ohio). Deconvolution of peaks in the DSC thermograms was performed using STAR software (Mettler Toledo, Columbus Ohio) to calculate the melting temperature of the double-crosslinked collagen material.

As shown in Table 3 below, collagen double crosslinked using methods of the present invention had increased melting temperatures compared to that of non-crosslinked or single-crosslinked collagen. For example, recombinant type III collagen crosslinked with a single crosslinking agent (GA or BDDE) displayed a melting temperature range from 68.0 to 68.4° C. (See Compositions “AY” and “BA” in Table 3.) However, recombinant type III collagen sequentially crosslinked with a first crosslinking agent (GA) and a second crosslinking agent (BDDE) displayed a melting temperature of 77.7° C. (See composition “BZ” in Table 3.) This data showed that the thermal stability (as determined by measurement of melting temperature) of collagen sequentially crosslinked with a first crosslinking agent and a second crosslinking agent was increased compared to collagen crosslinked with a single crosslinking agent alone. Further, since thermal stability is predictive of in vivo persistence and cross-linking efficiency, this data suggested that double-crosslinked collagen would be more persistent in vivo and further crosslinked than either single or non-crosslinked collagen.

TABLE 3

| | | First | Second | Melting |
|---|---|---|---|---|
| | Collagen | Crosslinking | Crosslinking | Temperature Tₘ, |
| Material | Type | Agent | Agent | (° C.) |
| I | III | GA (35 ppm) | GPGE (4%) | 76.3 |
| J | III | GA (35 ppm) | GPGE (4%) | 76.5 |
| K | III | GA (35 ppm) | GTGE (4%) | 66.9 |
| L | III | GA (35 ppm) | GTGE (4%) | 76.8 |
| M | III | GA (35 ppm) | EGDGE (4%) | 77.1 |
| N | III | GA (35 ppm) | EGDGE (4%) | 66.6 |
| O | III | GA (35 ppm) | — | 64.7 |
| AW | III | — | — | 46.0 |
| AX | III | GA (12 ppm) | — | 57.7 |
| AY | III | GA (35 ppm) | — | 68.0 |
| AZ | III | BDDE (4%) | — | 64.3 |
| BA | III | BDDE (4%) | — | 68.4 |
| BB | III | GA (12 ppm) | BDDE (2%) | 64.5 |
| BC | III | GA (12 ppm) | BDDE (2%) | 68.5 |
| BD | III | GA (12 ppm) | BDDE (2%) | 70.4 |
| BE | III | GA (12 ppm) | BDDE (2%) | 74.2 |
| BF | III | GA (12 ppm) | BDDE (4%) | 67.5 |
| BG | III | GA (12 ppm) | BDDE (4%) | 71.1 |
| BH | III | GA (12 ppm) | BDDE (4%) | 73.3 |
| BI | III | GA (12 ppm) | BDDE (4%) | 75.7 |
| BJ | III | GA (12 ppm) | BDDE (4%) | 70.8 |
| BK | III | GA (12 ppm) | BDDE (4%) | 74.8 |
| BL | III | GA (12 ppm) | BDDE (4%) | 74.5 |
| BM | III | GA (12 ppm) | BDDE (4%) | 74.3 |
| BN | III | GA (35 ppm) | BDDE (2%) | 72.4 |
| BO | III | GA (35 ppm) | BDDE (2%) | 72.7 |
| BP | III | GA (35 ppm) | BDDE (2%) | 73.8 |
| BQ | III | GA (35 ppm) | BDDE (2%) | 76.0 |
| BR | III | GA (35 ppm) | BDDE (2%) | 72.8 |
| BS | III | GA (35 ppm) | BDDE (2%) | 77.0 |
| BT | III | GA (35 ppm) | BDDE (2%) | 75.8 |
| BU | III | GA (35 ppm) | BDDE (2%) | 77.9 |
| BV | III | GA (35 ppm) | BDDE (4%) | 71.1 |
| BW | III | GA (35 ppm) | BDDE (4%) | 74.5 |
| BX | III | GA (35 ppm) | BDDE (4%) | 70.2 |
| BY | III | GA (35 ppm) | BDDE (4%) | 75.9 |
| BZ | III | GA (35 ppm) | BDDE (4%) | 77.7 |
| CA | III | GA (35 ppm) | BDDE (4%) | 72.8 |
| CB | III | GA (35 ppm) | BDDE (4%) | 77.0 |
| CC | III | GA (35 ppm) | BDDE (4%) | 73.8 |
| CD | III | GA (35 ppm) | BDDE (4%) | 75.8 |
| CE | III | GA (35 ppm) | BDDE (4%) | 75.5 |
| CF | III | GA (35 ppm) | BDDE (4%) | 75.5 |

See Table 1 above for reaction conditions (including pH, conc., and time) for each material.

### Example 4

### Extent of Crosslinking Measured by TNBS Assay

Free primary amino group content, as measured by a TNBS assay, has been used to determine the extent of crosslinking (Everaerts et al. (2004) Biomaterials 25:5523-30) of collagen materials. The free primary amino acid content (lysine residues) of collagen materials may be determined using 2,4,6-trinitrobenzene sulfonic acid (TNBS, Sigma-Aldrich, St. Louis Mo.). TNBS reagent has been used as a rapid and sensitive determination of free primary amino groups in proteinaceous materials. (Bubnis et al. (1992) Anal Biochem 207:129-33.) Primary amino groups, such as those found on lysine residues in collagen, form a chromogenic derivative (TNP-lysine) upon reaction with TNBS which may then be measured for optical density at 345 nm in a spectrophotometer. In this TNBS assay, the number of free lysine residues is used to measure the extent of crosslinking; a lower number of free lysine residues indicates a greater extent of crosslinking.

To examine the effect of methods of the present invention on the extent of collagen crosslinking, the following studies were performed. Samples of recombinant human type III collagen, single-crosslinked recombinant human type III collagen, and double-crosslinked recombinant human type III collagen were used in these studies. The crosslinking procedures and the preparation of lyophilized collagen materials were performed as described in Example 1 above.

TNBS reactions were performed as follows. Each collagen sample was suspended in 1 ml of 0.1M sodium carbonate, pH 9.0. To each collagen suspension, 1 ml of 0.5% TNBS was added and the mixture was allowed to react for 2 hours at 40° C. Following this reaction the collagen was solubilized with 3 ml of 6N HCl and incubated for 1.5 hours at 60° C. The solubilized collagen solution was then diluted with 5 ml deionized water to give a total of 10 ml solution. Of that solution, half (5 ml) was extracted three times with 10 ml of ethyl ether. Air was blown on the sample to remove residual ether in the aqueous collagen solution. The remaining aqueous collagen solution was diluted to 20 ml and the optical density of the solution was measured at 345 nm (OD₃₄₅) in a spectrophotometer (Molecular Devices, Sunnyvale Calif.). The amount of free lysine in moles per α-chain of recombinant human type-III collagen was determined from the absorbance reading using equations previously described. (Everaerts et al. (2004) Biomaterials 25:5523-30.)

As shown in Table 4 below, methods of the present invention increased the extent of crosslinking of recombinant human type III collagen, as measured by moles of free lysine. For example, the extent of crosslinking in recombinant type III collagen crosslinked with a single crosslinking agent (4% BDDE) was 69%. However, the sequential crosslinking of recombinant type III collagen with a first crosslinking agent (35 ppm GA) and a second crosslinking agent (4% BDDE) increased the extent of crosslinking to 89%. Thus, this data showed that the extent of crosslinking of collagen crosslinked with two crosslinking agents in a sequential manner was increased compared to collagen crosslinked with a single crosslinking agent alone. Further, since the extent of crosslinking is predictive of in vivo persistence, this data suggested that double-crosslinked recombinant collagen would be more persistent in vivo than single-crosslinked recombinant collagen.

TABLE 4

| Collagen | | Moles | Extent |
|---|---|---|---|
| Type | Crosslinking Agent(s) | Free Lysine | of Crosslinking (%) |
| III | none | 33.3 | — |
| III | 35 ppm GA | 18.9 | 43 |
| III | 1% BDDE | 16.5 | 50 |
| III | 2% BDDE | 14.4 | 57 |
| III | 4% BDDE | 10.2 | 69 |
| III | 6 ppm GA + 1% BDDE | 10.2 | 69 |
| III | 12 ppm GA + 1% BDDE | 10.2 | 69 |
| III | 35 ppm GA + 1% BDDE | 8.8 | 74 |
| III | 6 ppm GA + 2% BDDE | 6.7 | 80 |
| III | 12 ppm GA + 2% BDDE | 6.3 | 81 |
| III | 35 ppm GA + 2% BDDE | 6.3 | 81 |
| III | 6 ppm GA + 4% BDDE | 3.5 | 89 |
| III | 12 ppm GA + 4% BDDE | 3.5 | 89 |
| III | 35 ppm GA + 4% BDDE | 3.5 | 89 |

### Example 5

### Pendant Epoxy Group Assay

Active pendant epoxy groups can be introduced into the collagen molecules after crosslinking with epoxide crosslinking agents (e.g. BDDE). In the context of tissue implantation, these active pendant epoxy groups have the potential to react with surrounding tissue following implantation. (See Zeeman et al. (1999) Biomaterials 20:921-31.) Therefore, it is important to determine the content of the pendant epoxy groups in the final crosslinked collagen material.

To examine the effect of methods of the present invention on the content of pendant epoxy groups in the final collagen material, the following studies were performed. Single-crosslinked recombinant human type III collagen and double-crosslinked recombinant human type III collagen were produced according to the methods described above in Example 1 with the crosslinking agents shown below in Table 5. The double-crosslinked collagen materials were treated with and without an excess of glycine to quench the pendant epoxide groups. In brief, quenching of the double-crosslinked collagen materials was achieved by resuspending the collagen materials in 14 ml of 0.5 M glycine (Sigma-Aldrich, St. Louis Mo.) in 0.1 M NaHCO₃ buffer, pH 10. The collagen materials were quenched at 30° C. for 16 hours, washed with water, then freeze dried and tested for amine and pendant epoxy groups.

The content of pendant epoxy groups in the crosslinked collagens was determined according to the methods of Zeeman et al. (2000) J Biomed Mater Res 51:541-8. Briefly, the starting amine content of the collagen materials was determined by TNBS assay using methods described above in Example 4. Next, the crosslinked collagen materials were immersed in 2 ml of 0.5 M lysine methyl ester dihydrochloride prepared in 0.1 M NaHCO₃ buffer, pH 10. These mixtures were reacted at room temperature for 72 hours. Following the reaction, fibrils were washed with water and freeze dried. Approximately 4mg of the freeze dried material was examined for final amine group determination by TNBS assay as described above. The pendant epoxy content is equal to the final amine content of the collagen material after LM treatment minus the starting free amine content before the LM addition.

The results of these experiments are shown below in Table 5. Use of the epoxide crosslinking agent BDDE increased pendant epoxy groups in double-crosslinked collagen materials. As shown in Table 5 below, quenching of the double-crosslinked collagen materials with glycine reduced the content of pendant epoxy groups in these collagen materials. For example, the moles of pendant epoxy groups in recombinant type III collagen crosslinked with a first crosslinking agent (35 ppm GA) and a second crosslinking agent (4% BDDE) was 2.1. However, after quenching the same double-crosslinked collagen material with glycine the moles of pendant epoxy groups decreased to 0.8. These results showed that the amount of pendant epoxy groups in double-crosslinked collagen material prepared using methods of the present invention may be modified.

TABLE 5

| Collagen | | Quenched with | Moles |
|---|---|---|---|
| Type | Crosslinker | Glycine | Pendant Epoxy |
| III | none | No | — |
| III | 35 ppm GA | No | 0 |
| III | 6 ppm GA + 2.5% BDDE | No | 1.1 |
| III | 6 ppm GA + 2.5% BDDE | Yes | 1.0 |
| III | 35 ppm GA + 4% BDDE | No | 2.1 |
| III | 35 ppm GA + 4% BDDE | Yes | 0.8 |

### Example 6

### Collagenase Resistance of Double-Crosslinked Collagen

Bacterial Collagenase Digestion Assay:

Purified bacterial collagenase selectively degrades collagen but not proteins that lack the Gly-X-Y collagen repeat sequence. Bacterial collagenase is also capable of solubilizing insoluble collagen. Previously, it was shown that insoluble collagen fibrils are quantitatively degraded by this protease and that crosslinking the fibrils with gluteraldehyde decreased the rate of degradation. (McPherson et al. (1986) Journal of Biomedical Material Research 20:79-92.) Thus, bacterial collagenase may be used to determine the in vitro persistence of collagen compositions and as a model to predict in vivo persistence.

To examine the effect of the double crosslinking methods of the present invention on the resistance of collagen material to collagenase digestion in vitro, the following studies were performed. Recombinant human collagens were double-crosslinked using the methods described above in Example 1. The double-crosslinked collagen fibrils were transferred into water and freeze dried. Approximately 4 mg of double-crosslinked collagen materials were suspended in 0.5 ml of a bacterial collagenase (Collagenase Form III, Advanced Biofactures Corp., Lynbrook N.Y.) solution (96 U), corresponding to a ratio of approximately 24 U bacterial collagenase per milligram of double-crosslinked collagen. Each collagen-collagenase mixture was incubated at 37° C. for 24 hours and for 1 week. At the indicated times, samples were taken from each vial, centrifuged to pellet the remaining insoluble collagen fibrils, and the absorbance of a sample of the supernatant (final sample volume 100 μl) was measured for optical density at 225 nm (OD₂₂₅) in a spectrophotometer (Molecular Devices, Sunnyvale Calif.). In this assay, absorbance is used to measure the extent of collagen material digested; a higher absorbance indicates increased digestion of the collagen materials by the collagenase.

As shown in Table 6 below, double crosslinking collagen using methods of the present invention resulted in collagen materials having increased resistance to collagenase digestion. For Example, recombinant type III collagen crosslinked with a single crosslinking agent (BDDE) had an absorbance value of 0.4 after one week of digestion. However, recombinant type III collagen sequentially crosslinked with a first crosslinking agent (GA) and a second crosslinking agent (BDDE) had a lower absorbance value of 0.2. Since a higher absorbance value indicates increase digestion of the collagen by the collagenase, these results showed that double-crosslinked collagen was more resistant to collagenase digestion than single-crosslinked or non-crosslinked collagen. These results further indicated that double-crosslinked collagen is more persistent in this model of in vivo persistence.

TABLE 6

| | | First | Second | Digest | |
|---|---|---|---|---|---|
| | Collagen | Crosslinking | Crosslinking | 24 hr | Digest 1 wk |
| Comp. | Type | Agent | Agent | OD₂₂₅ | OD₂₂₅ |
| A | I | GA | BDDE | 0.04 | 0.07 |
| B | II | GA | BDDE | 0.04 | 0.07 |
| C | III | EDC | BDDE | 0.3 | 0.3 |
| D | III | EDC | BDDE | 0.4 | 0.4 |
| E | III | BDDE | — | 24 | 24 |
| F | III | BDDE | — | 24 | 24 |
| G | III | BDDE | — | 3 | 16 |
| H | III | BDDE | — | 0.4 | 0.4 |
| I | III | GA | GPGE | 0.11 | 0.19 |
| J | III | GA | GPGE | 0.45 | 1.08 |
| K | III | GA | GTGE | 0.11 | 0.15 |
| L | III | GA | GTGE | 0.19 | 0.62 |
| M | III | GA | EGDGE | 5.2 | 12.65 |
| N | III | GA | EGDGE | 5.1 | 11.87 |
| O | III | GA | — | 5.81 | 13.52 |
| P | I | FA | — | 24 | 24 |
| Q | I | FA | — | 24 | 24 |
| R | I | FA | — | 24 | 24 |
| S | I | FA | — | 12 | 24 |
| T | I | FA | — | 2 | 22 |
| U | I | FA | — | 0.6 | 7 |
| V | I | FA | BDDE | 0.24 | 0.56 |
| W | I | FA | BDDE | 0.13 | 0.37 |
| X | I | FA | BDDE | 0.08 | 0.10 |
| Y | I | GA | PPGDE | 0.05 | 0.06 |
| Z | I | GA | EGDGE | 0.06 | 0.05 |
| AA | I | GA | NPGDGE | 0.05 | 0.20 |
| AB | I | GA | TMPPGE | 0.04 | 0.05 |
| AC | I | GA | PEGDE | 0.04 | 0.05 |
| AD | III | GA | — | 20 | — |
| AE | III | GA | — | 20 | — |
| AF | III | GA | — | 0.3 | 23 |
| AG | III | GA | — | 0.3 | — |
| AH | III | GA | — | 0.3 | 2.5 |
| AI | III | GA | EDC | 1.5 | 1.8 |
| AJ | III | GA | EDC | 0.2 | 0.4 |
| AK | III | EDC | GA | 2.2 | 2.9 |
| AL | III | EDC | GA | 0.6 | 0.8 |
| AM | III | EDC | — | 26 | — |
| AN | III | EDC | — | 28 | — |
| AO | III | EDC | BDDE | 0.3 | 0.3 |
| AP | III | EDC | BDDE | 0.4 | 0.4 |
| AQ | I | EDC | BDDE | 0.24 | 0.23 |
| AR | I | EDC | BDDE | 0.36 | 0.35 |
| AS | III | GA | BDDE | 0.06 | 0.2 |
| AT | III | GA | BDDE | 0.12 | 0.16 |
| AU | III | GA | BDDE | 0.12 | 0.11 |
| AV | III | GA | BDDE | 0.11 | 0.09 |
| AW | III | — | — | 25.6 | 24.3 |

### Example 7

### Persistence of Double-Crosslinked Collagen in Vivo

In vivo persistence of implanted double-crosslinked recombinant human type III collagen was investigated as follows. Wistar rats (Charles River Laboratories, Wilmington Mass.) were shaved and an 8 cm×6 cm site for injection was marked the day prior to implantation. Purified single-crosslinked or double-crosslinked collagen materials were prepared as described above in Example 1. Single- and double-crosslinked collagen materials were resuspended in PBS to a final collagen concentration of 35 mg/ml.

Collagen implants were made by subcutaneous injection on the dorsal flank of 0.5 ml of a 35 mg/ml suspension of single-crosslinked or double-crosslinked recombinant human type III collagen suspension in PBS. Each collagen suspension was injected into the animals using a 1 cc syringe with a 30-gauge needle. Each animal received four separate injections of collagen material. Groups of 9, 10, or 11 animals per test material were analyzed for collagen implant persistence at 9 months and 16 months post implantation. At each time point, collagen implants were surgically removed and dissected free from surrounding tissue, weighed, and examined macroscopically for appearance and texture. Essentially no inflammatory or tissue response was observed following implantation of the single-crosslinked or double-crosslinked recombinant type III collagen materials.

As is standard in the art, the persistence of the collagen implants was evaluated by determining the number and wet weight of the original implants that were present at the injection sites at each of the time points. As shown in Table 7 below, the double-crosslinked recombinant type III collagen implants showed persistence markedly greater than those of single-crosslinked recombinant type III collagen. For example, 75% of the double-crosslinked recombinant human type III collagen implants were recovered at 16 months compared to only 33% of the single-crosslinked recombinant human type III collagen implants. Double-crosslinked collagen implants also showed increased wet weight of the remaining implants compared to single-crosslinked collagen implants.

TABLE 7

| | | Implant | | Implant |
|---|---|---|---|---|
| | Implants | Remaining | Implants | Remaining |
| | Recovered | (wet weight, | Recovered | (wet weight, |
| | (%) | %) | (%) | %) |
| Group | 9 month | 9 month | 16 month | 16 month |
| Collagen rhType III | 62 | 31 | 33 | 11 |
| (35 ppmGA only) | | | | |
| Collagen rhType III | 76 | 83 | 81 | 61 |
| (12 ppmGA + | | | | |
| 4% BDDE) | | | | |
| Collagen Type III-C | 80 | 75 | 95 | 92 |
| (12 ppmGA + | | | | |
| 4% BDDE) | | | | |
| Restylane ® | 96 | 108 | 94 | 93 |

These results showed that double-crosslinked collagen demonstrated improved persistence upon implantation, and is thus suitable for use in various tissue augmentation applications. These results also showed that double-crosslinked recombinant type III collagen was more persistent than single-crosslinked recombinant type III collagen; therefore, compositions and formulations containing double-crosslinked type III collagen material can provide unexpected benefits, e.g., enhanced persistence. In addition, these results demonstrated that a double-crosslinked recombinant type III collagen with six proline substitutions exhibited increased in vivo persistence compared to both single- and double-crosslinked recombinant human type III collagen.

### Example 8

### Decreased Immunogenicity of Double-Crosslinked Collagen in Vivo

Commercial bovine collagen dermal fillers have been shown to cause hypersensitivity reactions in 1-3% of patients. (Moody et al. (2001) Dermatol Surg 27:789-91.) In addition, increased anti-bovine collagen antibody titers following implantation have been observed as well as reports of connective tissue disease arising after bovine collagen injections. (Frank et al. (1991) Plast Reconstr Surg 87:1080-8). As a result of these adverse reactions, patients planning treatment with a bovine collagen product are required to take an allergy test approximately 1 month prior to the expected procedure date. Therefore, an implantable collagen material with decreased immunogenicity would be desirable.

Immunogenicity of collagen materials has been determined in experimental rat models. (Quteish et al. (1991) J Periodontal Res 26:114-21.) The immunogenicity of double-crosslinked collagen materials of the present invention was investigated in a rat model as follows. Double-crosslinked recombinant type III collagen was subcutaneously implanted in Wistar rats (Charles River Laboratories, Wilmington Mass.) as described above in Example 7. Serum from each animal was taken at 7, 9, and 16 months post implantation. Serum samples were analyzed for the presence of antibodies directed against human type III collagen using standard sandwich ELISA techniques (Quteish et al. (1991) J Periodontal Res 26:114-21) with a goat anti-human type III collagen antibody (Biodesign, cat #T33330G, Saco Me.).

As shown in Table 8 below, serum from animals implanted with single-crosslinked human type III collagen material contained antibody titers of antibodies directed against human type III collagen. This data indicated that the implanted single-crosslinked human type III collagen material was immunogenic to the animals (as evidenced by the presence of antibodies directed against human type III collagen in the animal's serum). In contrast, the implanted double-crosslinked type III collagen materials of the present invention showed no immunogenicity, as evidenced by the lack of any detectable antibody titer to human type III collagen, at any time point examined. These results indicated that double-crosslinked collagen implants are non-immunogenic in vivo. These results further indicated that double-crosslinked collagen implant materials are suitable for use in various medical procedures, including tissue augmentation procedures, in which low immunogenicity is desired.

TABLE 8

| | Human Type III | | |
|---|---|---|---|
| | collagen Antibody Titer | | |
| | | | Month |
| Group | Month 7 | Month 9 | 16 |
| Single-Crosslinked Collagen rhType III | 551 | 965 | 70 |
| (35 ppmGA only) | | | |
| Double-Crosslinked Collagen rhType III | ND | ND | ND |
| (12 ppmGA + 4% BDDE) | | | |
| Double-Crosslinked Collagen Type | ND | ND | ND |
| III-C (12 ppmGA + 4% BDDE) | | | |

ND—Not detected

Various modifications of the invention, in addition to those shown and described herein, will become apparent to those skilled in the art from the foregoing description. Such modifications are intended to fall within the scope of the appended claims.

All references cited herein are hereby incorporated herein by reference in their entirety.

What is claimed is:

1. A method for producing double-crosslinked collagen material comprising the steps of: (a) providing a collagen starting material, a first crosslinking agent, and a second crosslinking agent; (b) subjecting the collagen and the first crosslinking agent to a first crosslinking reaction, wherein the first crosslinking reaction is performed under reaction conditions that allow the first crosslinking reaction to occur, thereby obtaining a single-crosslinked collagen material; and (c) subjecting the single-crosslinked collagen material to a second crosslinking reaction using the second crosslinking agent, wherein the second crosslinking agent is not the same as the first crosslinking agent, and wherein the second crosslinking reaction is performed under reaction conditions that allow the second crosslinking reaction to occur, thereby obtaining a double-crosslinked collagen material.

2. The method according to claim 1, wherein the collagen starting material is collagen fibrils.

3. A method according to claim 1 or claim 2, wherein the collagen starting material is selected from the group consisting of type I, type II, type III, type V, or type XI collagen.

4. A method according to claim 3, wherein the collagen starting material is type III collagen.

5. A method according to any preceding claim, wherein the collagen starting material is recombinant collagen.

6. A method according to claim 5, wherein the collagen starting material is a single type of recombinant collagen.

7. A method according to any preceding claim, wherein the collagen starting material is free of endogenous crosslinks.

8. A method according to any preceding claim, wherein the collagen starting material is: a) type ill collagen having an amino acid sequence of SEQ ID NO:1; b) a collagen having an amino acid sequence of amino acid residue 168 to amino acid residue 1196 of SEQ ID NO:1; c) a collagen having an amino acid sequence of SEQ ID NO:2; d) a collagen having an amino acid sequence of from amino acid residue 38 to amino acid residue 1066 of SEQ ID NO:2; e) a collagen having an amino acid sequence of SEQ ID NO:2, wherein the amino acid sequence contains an isoleucine to proline substitution at amino acid residue 822 of SEQ ID NO:2; f) a collagen having an amino acid sequence of from amino acid residue 38 to amino acid residue 1066 of SEQ ID NO:2, wherein the amino acid sequence contains an isoleucine to proline substitution at amino acid residue 822 of SEQ ID NO:2; g) a collagen having an amino acid sequence of SEQ ID NO:2, wherein the amino acid sequence contains proline substitutions at amino acid residues 817, 820, 823, 826, and 829 of SEQ ID NO:2; h) a collagen having an amino acid sequence of from amino acid residue 38 to amino acid residue 1066 of SEQ ID NO:2, wherein the amino acid sequence contains proline substitutions at amino acid residues 817, 820, 823, 826, and 829 of SEQ ID NO:2; i) a collagen having an amino acid sequence of SEQ ID NO:2, wherein the amino acid sequence contains proline substitutions at amino acid residues 817, 820, 822, 823, 826, and 829 of SEQ ID NO:2; j) a collagen having an amino acid sequence of from amino acid residue 38 to amino acid residue 1066 of SEQ ID NO:2, wherein the amino acid sequence contains proline substitutions at amino acid residues 817, 820, 822, 823, 826, and 829 of SEQ ID NO:2; k) a collagen having an amino acid sequence of SEQ ID NO:2, wherein the amino acid sequence contains proline substitutions at amino acid residues 265, 300, 402, 414, 468, 471, 543, 567, 576, 603, 618, 693, 717, 738, and 900 of SEQ ID NO:2; or 1) a collagen having an amino acid sequence of from amino acid residue 38 to amino acid residue 1066 of SEQ ID NO:2, wherein the amino acid sequence contains proline substitutions at amino acid residues 265, 300, 402, 414, 468, 471, 543, 567, 576, 603, 618, 693, 717, 738, and 900 of SEQ ID NO:2.

9. A method according to any preceding claim, wherein a) the first crosslinking agent used in the first crosslinking reaction is an aldehyde compound and the second crosslinking agent used in the second crosslinking reaction is a carbodiimide or an epoxide compound; b) the first crosslinking agent used in the first crosslinking reaction is a carbodiimide compound and the second crosslinking agent used in the second crosslinking reaction is an epoxide or an aldehyde compound; or c) the first crosslinking agent used in the first crosslinking reaction is an epoxide compound and the second crosslinking agent used in the second crosslinking reaction is a carbodiimide or an aldehyde.

10. A method according to any preceding claim, wherein the first crosslinking agent is an aldehyde compound.

11. A method according to claim 10, wherein the first crosslinking agent is glutaraldehyde.

12. A method according to any preceding claim, wherein the second crosslinking agent is an epoxide compound.

13. A method according to claim 12, wherein the epoxide crosslinking agent is 1,4-butanediol diglycidyl ether (BDDE).

14. A method according to claim 12 or claim 13, wherein any pendant epoxide groups that remain in the double-crosslinked collagen material are quenched.

15. The method of claim 14, wherein the pendant epoxide groups are quenched by treating the double-crosslinked collagen material with an excess of glycine.

16. A method according to any preceding claim, wherein the crosslink initiated by the first crosslinking agent occurs by the reaction of the crosslinking agent with collagen α-amine groups of either lysine or hydroxylysine residues.

17. A method according to any preceding claim, wherein the crosslink initiated by the second crosslinking agent may also occur by the reaction of the crosslinking agent with collagen α-amine groups of either lysine or hydroxylysine residues.

18. A method according to any preceding claim, wherein the second crosslinking reaction is carried out at a basic pH.

19. A double-crosslinked collagen material produced by the method of any preceding claim.

20. A composition comprising the double-crosslinked collagen of claim 19.

21. A composition according to claim 20 which is implantable and/or injectable in to a human or animal body.

22. An implant comprising a composition according to claim 19.

23. The use of a double-crosslinked collagen material produced by the method of any of claims 1 to 18 in the preparation of a product for pharmaceutical, cosmetic, or medical use.

24. A double-crosslinked collagen material produced by the method of any of claims 1 to 18 for use in therapy or surgery.

25. A double-crosslinked collagen material produced by the method of any of claims 1 to 18 for use in tissue augmentation or repair.

26. A cosmetic procedure comprising injecting or implanting a double-crosslinked collagen material produced by the method of any of claims 1 to 18 into the skin or dermis of a subject.
